(19) United States
(12) Patent Application Publication
(10) Pub. No.: US 2021/0196808 A1
(43) Pub. Date: Jul. 1, 2021

(54) CANCER VACCINE

(71) Applicant: UNIVERSITY COLLEGE CARDIFF CONSULTANTS LTD, Cardiff, South Glamorgan (GB)

(72) Inventors: Andrew GODKIN, Cardiff (GB); Awen GALLIMORE, Cardiff (GB); Martin SCURR, Cardiff (GB)

(73) Assignee: University College Cardiff Consultants Limited, Cardiff, South Glamorgan (GB)

(21) Appl. No.: 17/055,293

(22) PCT Filed: May 14, 2019
(86) PCT No.: PCT/GB2019/051309

§ 371 Date: Nov. 13, 2020

(30) Foreign Application Priority Data
May 15, 2018 (GB) 1807831.1

(51) Int. Cl. A61K 39/00; A61P 35/00

(52) U.S. Cl. CPC A61K 39/001176; A61P 35/00

(57) ABSTRACT

The present disclosure relates to an immunogenic agent comprising Dna J heat shock protein family (Hsp40) member B7 or an immunogenic fragment thereof; a DNA vaccine comprising a nucleic acid encoding said protein or at least one immunogenic fragment thereof; a pharmaceutical composition or vector or DNA vaccine for use in the treatment of cancer; and a method of treating cancer comprising the use of said immunogenic agent or pharmaceutical composition or vector or DNA vaccine.

### TECHNICAL FIELD

The present disclosure relates to an immunogenic agent comprising DnaJ heat shock protein family (Hsp40) member B7 or an immunogenic fragment thereof; a DNA vaccine comprising a nucleic acid encoding said protein or at least one immunogenic fragment thereof; a pharmaceutical composition or vector or DNA vaccine for use in the treatment of cancer; and a method of treating cancer comprising the use of said immunogenic agent or pharmaceutical composition or vector or DNA vaccine.

### BACKGROUND

Despite understandable excitement surrounding results from clinical studies of immunotherapies for cancer, actual outcomes are disappointing. Many upregulated tumour associated antigen (TAA) targets for immunotherapy are often expressed to some extent in or on healthy tissue, e.g. the autoantigen carcinoembryonic antigen (CEA), and so directing immune responses against such antigens can lead to unwanted side-effects. Indeed, we have found T cell responses to CEA are associated with early disease relapse in patients treated for colorectal cancer (CRC). Thus, identification of useful TAAs that can be targeted by immunotherapy is a balance between: i) tumour expression; ii) the levels of expression of the same antigen in healthy tissue; and iii) controlling antigen-specific immunosuppressive responses driven by the same antigens. The challenge is further complicated by T cell cross reactivity which can result in off-target effects in distant tissue with potentially fatal consequences.

Whilst immunotherapies targeting neoepitopes hold promise as they are likely to differ sufficiently from self-antigens to ensure no cross reactivity, they are highly focused at the level of the individual and so prohibitively expensive to develop. For therapies relevant to the wider population such as cancer vaccines, immune mobilising monoclonal T-cell receptors Against Cancer (ImmTACs) and Chimeric Antigen Receptor T (CAR-T) cells, antigens must be broadly expressed in the same tumour types of multiple individuals and present at minimal levels in healthy tissue. Ideally, discovery pipelines would involve the large-scale analysis of TAA candidates followed by selection based on immunogenicity and tissue specific expression. Candidates that fit these criteria could be explored further as suitable cancer vaccinations.

In the context of CRC, known TAAs include CEA, GUCY2C, 5T4, MAGE antigens and Her-2, with several large investigations into cancer-testis antigen expression panels that have resulted in the identification of novel antigens; but the problem with these antigens is that they are often expressed in only a limited proportion of tumours. Of these antigens, CEA, GUCY2C and 5T4 have progressed towards pre-clinical and clinical studies.

There is therefore a need for new cancer vaccines that can be administered to a broad, ideally HLA-disparate population. The ideal vaccine would induce or boost T cell responses to target antigens expressed only in transformed cancer cells, but not normal cells; this is important to avoid off-target toxicity. Genomic analysis of paired tumour and healthy tissue in theory facilitates TAA identification but in practice appears to be limited by the diversity of cellular input in each sequencing sample.

The increasing use of RNA sequencing (RNA-seq) in differential expression analysis provides a useful methodology to initiate TAA discovery pipelines. However, for the colon, a mixture of immune cells, epithelium and stroma tends to complicate investigated expression profiles, thus hindering the identification of significantly and differently expressed genes, especially when one also considers that tumour immune infiltrate varies between individuals and tumour location.

The purification of epithelial and tumour cells prior to RNA seq analysis is a novel way for overcoming tissue heterogeneity. In this study, we used Epithelial cell adhesion molecule (EpCAM), i.e. a transmembrane glycoprotein mediating Ca2+-independent homotypic cell-cell adhesion in epithelia to aid purification. EpCAM purification of tumour and healthy colonic epithelium at two sites improved the resolution between tumour and healthy cell expression profiles and thus aided the identification of differentially expressed genes (DEG). Gene lists were created based on expression profiles between all tissues, and significant expression levels were established in a DESEQ2 comparison analysis. These lists were analysed, and several genes selected for further investigation as vaccine candidates. Immunogenic analysis and tissue expression of the protein products of these genes in healthy tissues were used to select the best candidate for future CRC immunotherapy. Several targets exhibited significant immunogenicity with respect to control antigens; but of these, one marker, DnaJ heat shock protein family (Hsp40) member B7 (DNAJB7) demonstrated the most favourable expression profile based on immunohistochemistry data of healthy and cancerous tissue. Further, we identified that all donors have the capability to mount T cell responses against DNAJB7. This protein antigen therefore harbours the potential to be used as a vaccine target in in future cancer therapeutic and prophylactic treatment strategies.

### STATEMENTS OF INVENTION

According to a first aspect of the invention there is provided an immunogenic agent for use as a cancer vaccine comprising or consisting of DnaJ heat shock protein family (Hsp40) member B7 (termed DNAJB7) or at least one immunogenic fragment thereof.

Reference herein to DNAJB7 is to a protein belonging to the evolutionarily conserved DNAJ/HSP40 family of proteins, which regulate molecular chaperone activity by stimulating ATPase activity. As is known by those skilled in the art, DNAJ proteins may have up to 3 distinct domains: a conserved 70-amino acid J domain, usually at the N terminus; a glycine/phenylalanine (G/F)-rich region; and a cysteine-rich domain containing 4 motifs resembling a zinc finger domain.

In a preferred embodiment of the invention said DNAJB7 is human DNAJB7.

Preferably said DNAJB7 is represented by the amino acid sequence set forth in SEQ ID NO: 31 or a sequence having at least 85%, 86%, 87%, 88%, 89%, 90%, 91%, 92%, 93%, 94%, 95%, 96%, 97%, 98% or 99% identity therewith.

Reference herein to an immunogenic fragment is to a part of DNAJB7 that can elicit an immune response when used in vivo; this response may be measured or determined using known tests such as those described herein. One test which may be used, but not exclusively, is whether the fragment can cause a tumour to be recognised and acted upon by components of the immune system. Advantageously, it has been found that these immunogenic fragments from portions of the full length DNAJB7 peptide, spanning from close to the C-terminus to the N-terminus i.e. the entire length of peptide, can elicit a response.

In a preferred embodiment of the invention said at least one fragment is 5-30 amino acids in length; preferably said at least one fragment is 8-25 amino acids in length. Most preferably said at least one fragment is 19 amino acids in length.

In a preferred embodiment of the invention said at least one DNAJB7 fragment comprises or consists of an amino acid sequence selected from at least one of the following groups:

a)
(SEQ ID NO: 1)
MVDYYEVLGLQRYASPEDIK;
(SEQ ID NO: 2)
QRYASPEDIKKAYHKVALKW;
(SEQ ID NO: 3)
KAYHKVALKWHPDKNPENKE;
(SEQ ID NO: 4)
HPDKNPENKEEAERKFKEVA;
(SEQ ID NO: 5)
EAERKFKEVAEAYEVLSNDE;
(SEQ ID NO: 6)
EAYEVLSNDEKRDIYDKYGT;
(SEQ ID NO: 7)
KRDIYDKYGTEGLNGGGSHF;
(SEQ ID NO: 8)
EGLNGGGSHFDDECEYGFTF;
(SEQ ID NO: 9)
DDECEYGFTFHKPDDVFKEI;
(SEQ ID NO: 10)
HKPDDVFKEIFHERDPFSFH;
(SEQ ID NO: 11)
FHERDPFSFHFFEDSLEDLL;
(SEQ ID NO: 12)
FFEDSLEDLLNRPGSSYGNR;
(SEQ ID NO: 13)
NRPGSSYGNRNRDAGYFFST;
(SEQ ID NO: 14)
NRDAGYFFSTASEYPIFEKF;
(SEQ ID NO: 15)
ASEYPIFEKFSSYDTGYTSQ;
(SEQ ID NO: 16)
SSYDTGYTSQGSLGHEGLTS;
(SEQ ID NO: 17)
GSLGHEGLTSFSSLAFDNSG;
(SEQ ID NO: 18)
FSSLAFDNSGMDNYISVTTS;
(SEQ ID NO: 19)
MDNYISVTTSDKIVNGRNIN;
(SEQ ID NO: 20)
DKIVNGRNINTKKIIESDQE;
(SEQ ID NO: 21)
TKKIIESDQEREAEDNGELT;
(SEQ ID NO: 22)
REAEDNGELTFFLVNSVANE;
(SEQ ID NO: 23)
FFLVNSVANEEGFAKECSWR;
(SEQ ID NO: 24)
EGFAKECSWRTQSFNNYSPN;
(SEQ ID NO: 25)
TQSFNNYSPNSHSSKHVSQY;
(SEQ ID NO: 26)
SHSSKHVSQYTFVDNDEGGI;
(SEQ ID NO: 27)
TFVDNDEGGISWVTSNRDPP;
(SEQ ID NO: 28)
SWVTSNRDPPIFSAGVKEGG;
(SEQ ID NO: 29)
IFSAGVKEGGKRKKKKRKEV;
and
(SEQ ID NO. 30)
KRKKKKRKEVQKKSTKRNC;

or
b) a fragment that has at least 85% sequence identity with any one or more of the sequences in group a) and, in ascending order of preference, at least 86%, 87%, 88%, 89%, 90%, 91%, 92%, 93%, 94%, 95%, 96%, 97%, 98% or 99% sequence identity with any one or more of the sequences in group a); or
c) a fragment that is a variant of any one or more of the sequences in group a) and/or group b) wherein said variant is modified by the addition, deletion or substitution of one or more amino acid residues in any one or more of the above sequences and, ideally but not always, wherein said variant fragment retains or has enhanced or comparable immunogenicity when compared to the immunogenicity of any one or more of the variants in group a) and/or group b).

As will be appreciated by those skilled in the art, measurement and/or comparison of immunogenicity can be carried out by any means known to those skilled in the art such as, but not limited to, in vitro FluoroSpot assays whereby peripheral blood mononuclear cell(s) is/are cultured in the presence of DNAJB7 protein or peptides derived therefrom, and T cell cytokine production in response to these peptides is measured.

As is known in the art, a variant polypeptide may differ in amino acid sequence by one or more substitutions, additions, deletions or truncations that may be present in any combination. Among preferred variants are those that vary from a reference polypeptide by conservative amino acid substitutions. Such substitutions are those that substitute a given amino acid for another amino acid of like characteristics. For example, charged amino acid residues include lysine (+), arginine (+), histidine (+), aspartate (−) and glutamate (−); polar amino acids include serine, threonine, asparagine, glutamine, and tyrosine whereas the hydrophobic amino acids include alanine, valine, leucine, isoleucine, proline, phenylalanine, tryptophan, cysteine and methionine. Generally, glycine is often found at the surface of proteins, within a loop- or coil region, providing high flexibility to the polypeptide chain at these locations. This suggests that it is rather hydrophilic. Proline, on the other hand, is generally non-polar and is mostly found buried inside the protein, although similarly to glycine, it is often found in loop regions. In contrast to glycine, proline provides rigidity to the polypeptide chain by imposing certain torsion angles on the segment of the structure. Glycine and proline are often highly conserved within a protein family since they are essential for the conservation of a particular protein fold.

In addition, in the following non-limiting groups of amino acids, each amino acid within each group are considered conservative replacements for one another: a) alanine, serine, and threonine; b) glutamic acid and aspartic acid; c) asparagine and glutamine d) arginine, histidine and lysine; e) isoleucine, leucine, methionine and valine and f) phenylalanine, tyrosine and tryptophan. Most highly preferred are variants that retain or have enhanced biological function, or immunogenicity, having regard to the reference polypeptide from which it varies.

In a preferred embodiment of the invention said at least one DNAJB7 fragment is represented by an amino acid sequence selected from the group comprising or consisting of:

a)
(SEQ ID NO: 23)
FFLVNSVANEEGFAKECSWR;
(SEQ ID NO: 11)
FHERDPFSFHFFEDSLEDLL;
(SEQ ID NO: 30)
KRKKKKRKEVQKKSTKRNC;
(SEQ ID NO: 26)
SHSSKHVSQYTFVDNDEGGI;
((SEQ ID NO: 8)
EGLNGGGSHFDDECEYGFTF;
(SEQ ID NO: 13)
NRPGSSYGNRNRDAGYFFST;
(SEQ ID NO: 14)
NRDAGYFFSTASEYPIFEKF;
(SEQ ID NO: 19)
MDNYISVTTSDKIVNGRNIN;
and
(SEQ ID NO: 27)
TFVDNDEGGISWVTSNRDPP;

or

b) a fragment that has at least 85% sequence identity with any one or more of the sequences in group a) and, in ascending order of preference, at least 86%, 87%, 88%, 89%, 90%, 91%, 92%, 93%, 94%, 95%, 96%, 97%, 98% or 99% sequence identity with any one or more of the sequences in group a); or

c) a fragment that is a variant of any one or more of the sequences in group a) and/or b) wherein said variant is modified by the addition, deletion or substitution of one or more amino acid residues in any one or more of the above sequences and, ideally but not always, wherein said variant fragment retains or has enhanced or comparable immunogenicity when compared to the immunogenicity of any one or more of the variants in group a) and/or group b).

According to a further aspect of the invention there is provided a vector or DNA vaccine comprising a nucleic acid molecule encoding said DnaJ heat shock protein family (Hsp40) member B7 (termed DNAJB7), or at least one fragment thereof, as herein disclosed.

In a preferred embodiment of the invention said nucleic acid molecule is part of, or provided in, an expression vector adapted to express said DNAJB7, or at least one of said fragments thereof.

Typically said adaptation includes, the provision of at least one transcription control sequences (e.g. at least one promoter sequence) which mediate(s) said expression. Preferably, the promoter is/are cell/tissue specific and more ideally still adapted for inducible or constitutive expression of said DNAJB7, or at least one fragment thereof.

In certain embodiments, said nucleic acid molecule encodes the whole of said DNAJB7 and/or a number of fragments thereof.

Those skilled in the art will appreciate that the term promoter includes the following features, which are provided by example only, and not by way of limitation: at least one enhancer element which is a cis acting nucleic acid sequences often found 5′ to the transcription initiation site of a gene (enhancers can also be found 3′ to a gene sequence or even located in intronic sequences and is therefore position independent) that functions to increase the rate of transcription of the gene to which the enhancer is linked. Further, enhancer activity is responsive to trans acting transcription factors (such as polypeptides) which have been shown to bind specifically to enhancer elements. The binding/activity of transcription factors (please see Eukaryotic Transcription Factors, by David S Latchman, Academic Press Ltd, San Diego) is responsive to a number of environmental cues which include, by example and not by way of limitation, intermediary metabolites (e.g. glucose, lipids), environmental effectors (e.g. light, heat,).

Promoter elements also include a TATA box and an RNA polymerase initiation selection (RIS) sequence which function to provide a site of transcription initiation. These sequences also bind polypeptides which function, inter alia, to facilitate transcription initiation selection by RNA polymerase.

Adaptations to the vector also include the provision of selectable markers and autonomous replication sequences which facilitate the maintenance of said vector in either a eukaryotic cell or prokaryotic host. Vectors which are maintained autonomously are referred to as episomal vectors and are included within the scope of the invention.

Further adaptations to the vector included within the scope of the invention, which facilitate the expression of vector encoded genes, include transcription termination/polyadenylation sequences. This or these features also includes the provision of internal ribosome entry sites (IRES) which function to maximise expression of vector encoded genes arranged in bicistronic or multi-cistronic expression cassettes.

Further adaptations to the vector included within the scope of the invention are expression control sequences, such as Locus Control Regions (LCRs). These are regulatory elements which confer position-independent, copy number-dependent expression to linked genes when assayed as transgenic constructs in mice. LCRs include regulatory elements that insulate transgenes from the silencing effects of adjacent heterochromatin, Grosveld et al., Cell (1987), 51: 975-985.

The above adaptations are well known in the art. Indeed, there is a significant amount of published literature with respect to expression vector construction and recombinant DNA expression techniques in general. Please see, Sambrook et al (1989) Molecular Cloning: A Laboratory Manual, Cold Spring Harbour Laboratory, Cold Spring Harbour, N.Y. and references therein; Marston, F (1987) DNA Cloning Techniques: A Practical Approach Vol III IRL Press, Oxford UK; DNA Cloning: F M Ausubel et al, Current Protocols in Molecular Biology, John Wiley & Sons, Inc.(1994). Any one or more of these known techniques may be used when working the invention, provided the final vector or construct contains a nucleic acid molecule encoding DNAJB7, or at least one fragment thereof, which can be expressed by said vector in culture or a host environment.

According to a further aspect of the invention there is provided a pharmaceutical composition comprising said immunogenic agent or vector or DNA vaccine of the invention.

According to a further aspect of the invention there is provided at least one immunogenic agent or vector or DNA vaccine or pharmaceutical composition containing or encoding DNAJB7, or at least one fragment thereof, for use in the treatment of cancer.

According to a further aspect of the invention there is provided at least one immunogenic agent or vector or DNA vaccine or pharmaceutical composition containing or encoding DNAJB7, or at least one fragment thereof, for use in the manufacture of a medicament to treat cancer.

As used herein, the term “cancer” refers to cells having the capacity for autonomous growth, i.e., an abnormal state or condition characterized by uncontrolled cell proliferation. The term is meant to include all types of cancerous growths or oncogenic processes, metastatic tissues or malignantly transformed cells, tissues, or organs, irrespective of histopathologic type or stage of invasiveness.

Most preferably the cancer referred to herein includes any one or more of the following cancers: nasopharyngeal cancer, synovial cancer, hepatocellular cancer, renal cancer, cancer of connective tissues, melanoma, lung cancer, bowel cancer, colon cancer, rectal cancer, colorectal cancer, brain cancer, throat cancer, oral cancer, liver cancer, bone cancer, pancreatic cancer, choriocarcinoma, gastrinoma, pheochromocytoma, prolactinoma, T-cell leukemia/lymphoma, tonsil, spleen, neuroma, von Hippel-Lindau disease, Zollinger-Ellison syndrome, adrenal cancer, anal cancer, bile duct cancer, bladder cancer, ureter cancer, glioma, oligodendroglioma, neuroblastoma, meningioma, spinal cord tumor, bone cancer, osteochondroma, chondrosarcoma, Ewing's sarcoma, cancer of unknown primary site, carcinoid, carcinoid of gastrointestinal tract, fibrosarcoma, breast cancer, muscle cancer, Paget's disease, cervical cancer, rectal cancer, esophagus cancer, gall bladder cancer, cholangioma cancer, head cancer, eye cancer, nasopharynx cancer, neck cancer, kidney cancer, Wilms' tumor, liver cancer, Kaposi's sarcoma, prostate cancer, testicular cancer, Hodgkin's disease, non-Hodgkin's lymphoma, skin cancer, mesothelioma, myeloma, multiple myeloma, ovarian cancer, endocrine pancreatic cancer, glucagonoma, parathyroid cancer, penis cancer, pituitary cancer, soft tissue sarcoma, retinoblastoma, small intestine cancer, stomach cancer, thymus cancer, thyroid cancer, trophoblastic cancer, hydatidiform mole, uterine cancer, endometrial cancer, vagina cancer, vulva cancer, acoustic neuroma, mycosis fungoides, insulinoma, carcinoid syndrome, somatostatinoma, gum cancer, heart cancer, lip cancer, meninges cancer, mouth cancer, nerve cancer, palate cancer, parotid gland cancer, peritoneum cancer, pharynx cancer, pleural cancer, salivary gland cancer, tongue cancer and tonsil cancer.

More preferably still, said cancer is selected from the group comprising the following cancers: colorectal cancer, thyroid, lymphoma, lung, liver, pancreatic, carcinoid, head & neck, stomach, urothelial, prostate, testis, endometrial, glioma, breast, cervical, ovarian, melanoma, pancreatic, liver, and renal cancers.

Yet more preferably still, said cancer is colorectal cancer, head and neck squamous cell carcinoma or liver cancer.

According to a further aspect of the invention there is provided a method of vaccinating a subject suffering from or having a predisposition for cancer comprising administering an effective amount of the immunogenic agent, vector, DNA vaccine or pharmaceutical composition according to the invention to said subject.

Most preferably the cancer referred to herein includes any one or more of the following cancers: nasopharyngeal cancer, synovial cancer, hepatocellular cancer, renal cancer, cancer of connective tissues, melanoma, lung cancer, bowel cancer, colon cancer, rectal cancer, colorectal cancer, brain cancer, throat cancer, oral cancer, liver cancer, bone cancer, pancreatic cancer, choriocarcinoma, gastrinoma, pheochromocytoma, prolactinoma, T-cell leukemia/lymphoma, tonsil, spleen, neuroma, von Hippel-Lindau disease, Zollinger-Ellison syndrome, adrenal cancer, anal cancer, bile duct cancer, bladder cancer, ureter cancer, glioma, oligodendroglioma, neuroblastoma, meningioma, spinal cord tumor, bone cancer, osteochondroma, chondrosarcoma, Ewing's sarcoma, cancer of unknown primary site, carcinoid, carcinoid of gastrointestinal tract, fibrosarcoma, breast cancer, muscle cancer, Paget's disease, cervical cancer, colon cancer, rectal cancer, esophagus cancer, gall bladder cancer, cholangioma cancer, head cancer, eye cancer, nasopharynx cancer, neck cancer, kidney cancer, Wilms' tumor, liver cancer, Kaposi's sarcoma, prostate cancer, testicular cancer, Hodgkin's disease, non-Hodgkin's lymphoma, skin cancer, mesothelioma, myeloma, multiple myeloma, ovarian cancer, endocrine pancreatic cancer, glucagonoma, pancreatic cancer, parathyroid cancer, penis cancer, pituitary cancer, soft tissue sarcoma, retinoblastoma, small intestine cancer, stomach cancer, thymus cancer, thyroid cancer, trophoblastic cancer, hydatidiform mole, uterine cancer, endometrial cancer, vagina cancer, vulva cancer, acoustic neuroma, mycosis fungoides, insulinoma, carcinoid syndrome, somatostatinoma, gum cancer, heart cancer, lip cancer, meninges cancer, mouth cancer, nerve cancer, palate cancer, parotid gland cancer, peritoneum cancer, pharynx cancer, pleural cancer, salivary gland cancer, tongue cancer and tonsil cancer.

More preferably still, said cancer is selected from the group comprising the following cancers: colorectal cancer, thyroid, lymphoma, lung, liver, pancreatic, carcinoid, head & neck, stomach, urothelial, prostate, testis, endometrial, glioma, breast, cervical, ovarian, melanoma, pancreatic, liver, and renal cancers.

Yet more preferably still, said cancer is colorectal cancer, head and neck squamous cell carcinoma or liver cancer.

In the claims which follow and in the preceding description of the invention, except where the context requires otherwise due to express language or necessary implication, the word “comprises”, or variations such as “comprises” or “comprising” is used in an inclusive sense i.e. to specify the presence of the stated features but not to preclude the presence or addition of further features in various embodiments of the invention.

All references, including any patent or patent application, cited in this specification are hereby incorporated by reference. No admission is made that any reference constitutes prior art. Further, no admission is made that any of the prior art constitutes part of the common general knowledge in the art.

Preferred features of each aspect of the invention may be as described in connection with any of the other aspects.

Other features of the present invention will become apparent from the following examples. Generally speaking, the invention extends to any novel one, or any novel combination, of the features disclosed in this specification (including the accompanying claims and drawings). Thus, features, integers, characteristics, compounds or chemical moieties described in conjunction with a particular aspect, embodiment or example of the invention are to be understood to be applicable to any other aspect, embodiment or example described herein, unless incompatible therewith.

Moreover, unless stated otherwise, any feature disclosed herein may be replaced by an alternative feature serving the same or a similar purpose.

Throughout the description and claims of this specification, the singular encompasses the plural unless the context otherwise requires. In particular, where the indefinite article is used, the specification is to be understood as contemplating plurality as well as singularity, unless the context requires otherwise.

An embodiment of the present invention will now be described by way of example only with reference to the following wherein:

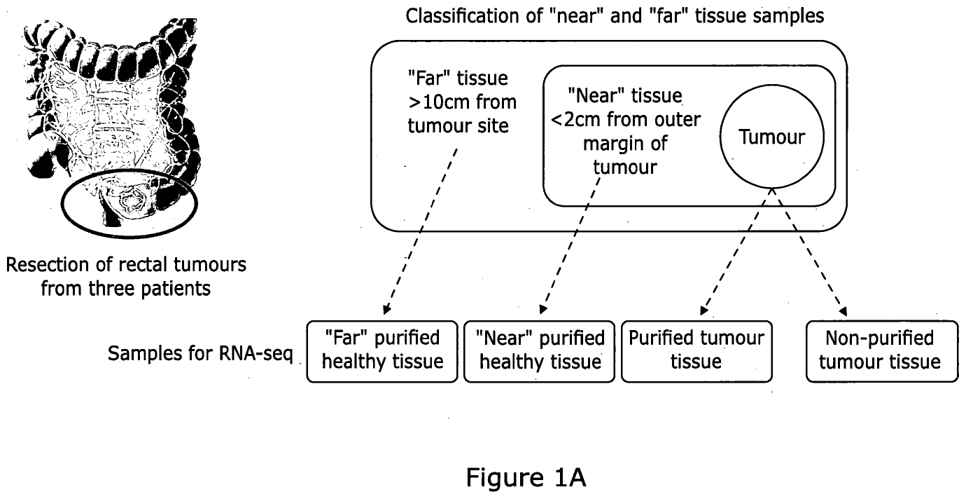

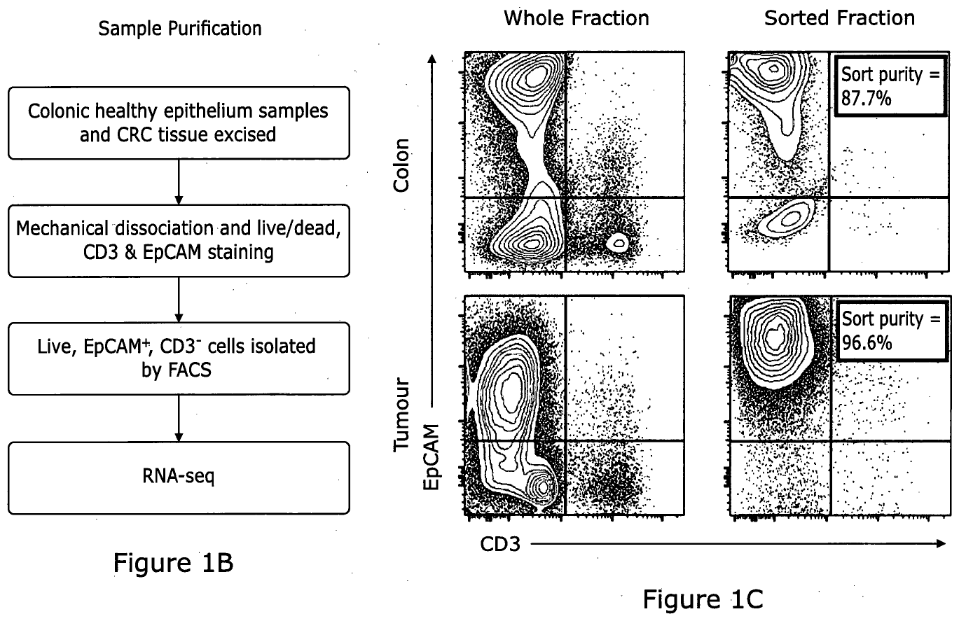

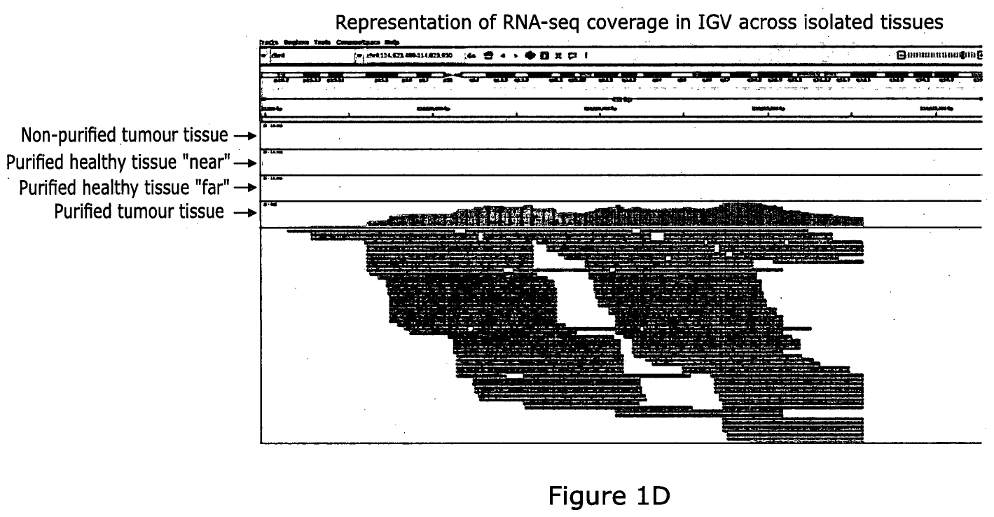

FIG. 1. Isolation of epithelial and tumour cells by EpCAM-sorting prior to RNA-seq. (A) Schematic of tumour and healthy tissue resection taken at two distances from the tumour site. Samples were taken from rectal tumour of three patients. (B) Sample processing and purification flow chart. (C) Flow cytometry gating for EpCAM⁺ and CD3⁻ purification, pre and post cell sorting. (D) Example of reads across four RNA-seq datasets in healthy tissue “near” and “far” compared to non-purified and purified tumour tissue. Image was taken from the integrative genomics viewer (Broad institute). Four arrows indicate coverage bar, while bottom box show aligned reads in one set of patient samples.

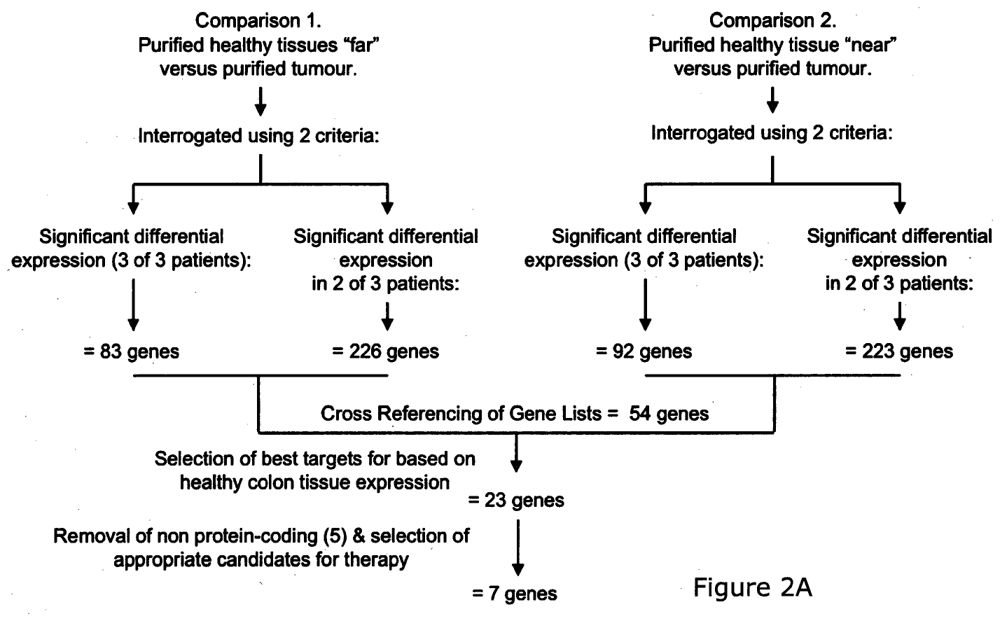

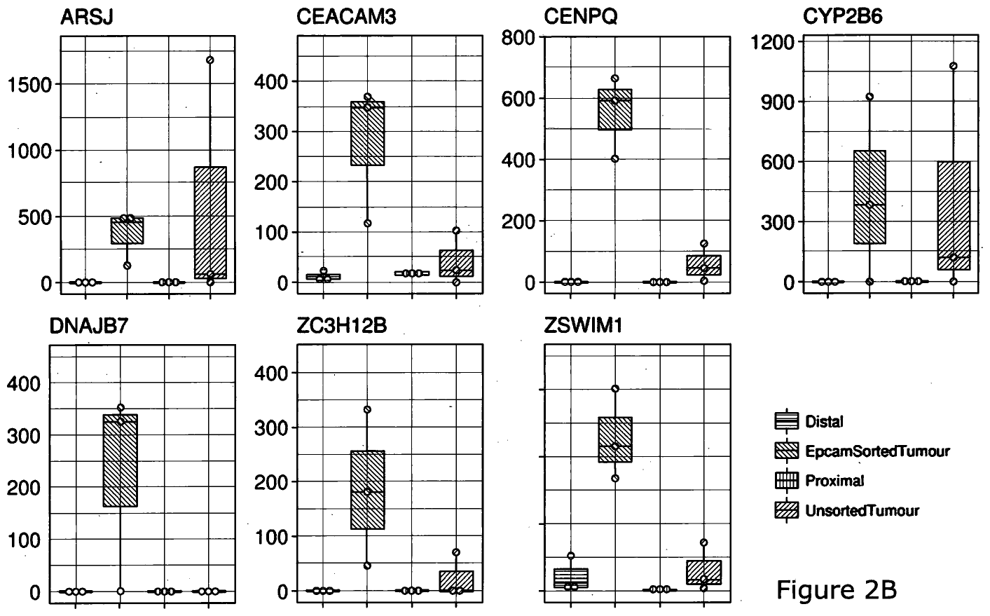

FIG. 2. Identification of candidates for further investigation based on differential expression analysis. (A) Workflow for obtaining gene lists of differentially expressed genes based on two comparisons (purified tumour versus purified healthy colon “far,” and purified tumour versus purified healthy colon “near,” left-hand side and right-hand side respectively). Gene lists were obtained from significantly differentially expressed genes across all three patients and separately in two of three patients. These were aligned, and cross referenced between “far” and “near” comparisons to give a smaller gene list which was further reduced based on expression in healthy tissue, and finally suitability for further investigation. (B) Normalised counts for each of the seven genes selected for further analysis. Counts are shown for each of the four conditions as box plots representing all three patients.

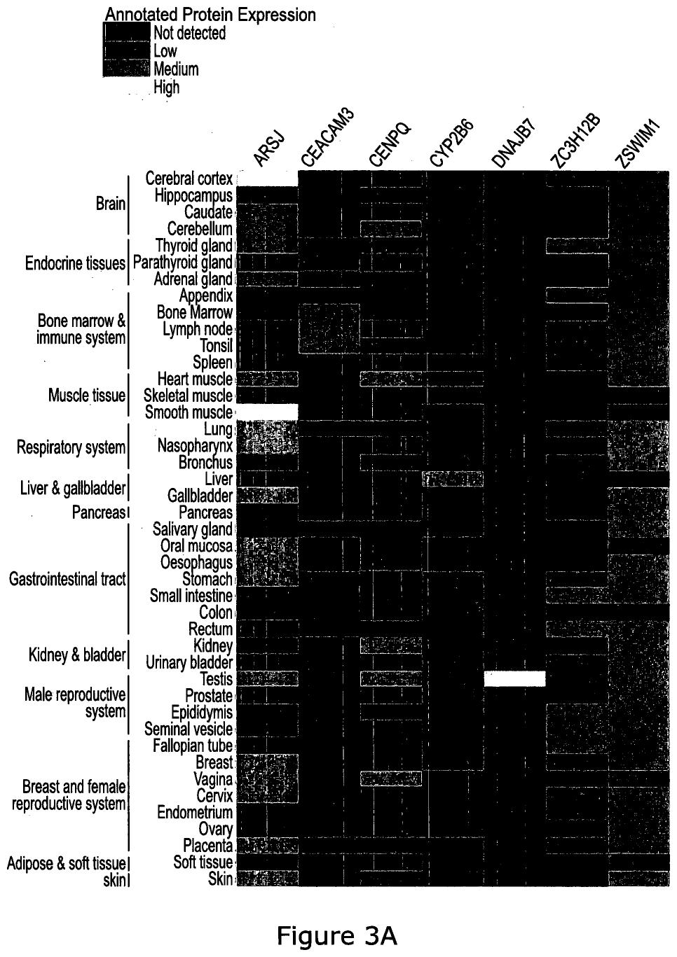

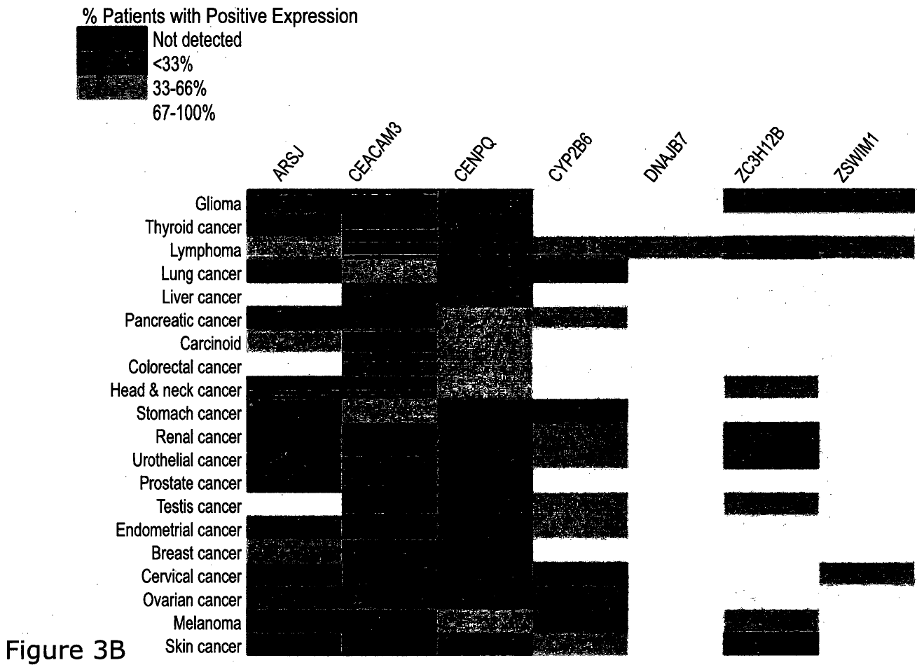

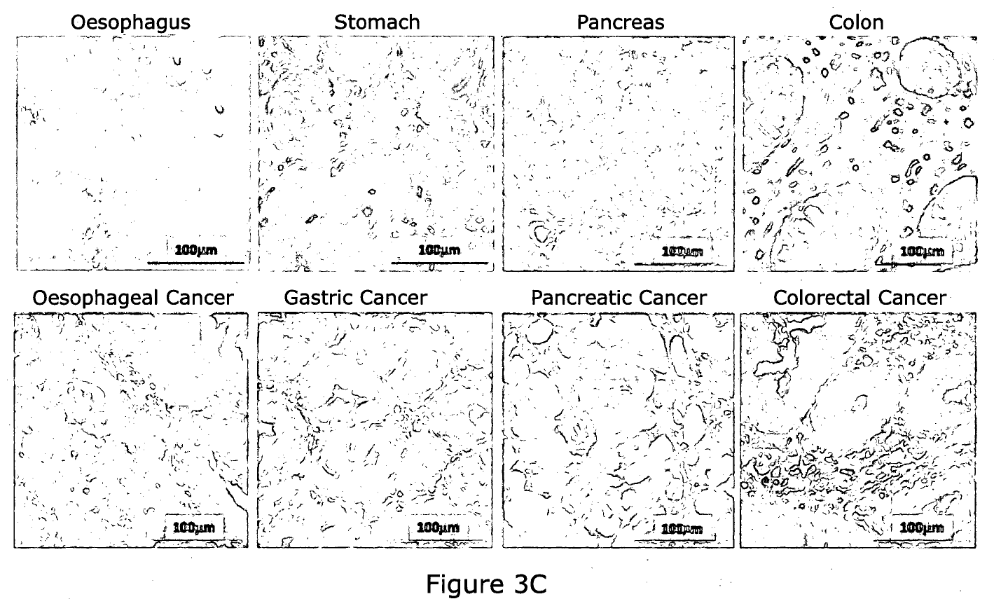

FIG. 3. Expression profile of candidate tumour antigens in healthy tissue and cancer. Utilising the Protein Atlas, the protein expression of the seven identified TAAs was evaluated in a range of healthy tissues (A) and tumour types by location (B). Immunohistochemistry revealed DNAJB7 expression to be confined to tumour samples, examples shown of staining in gastrointestinal tumours (C).

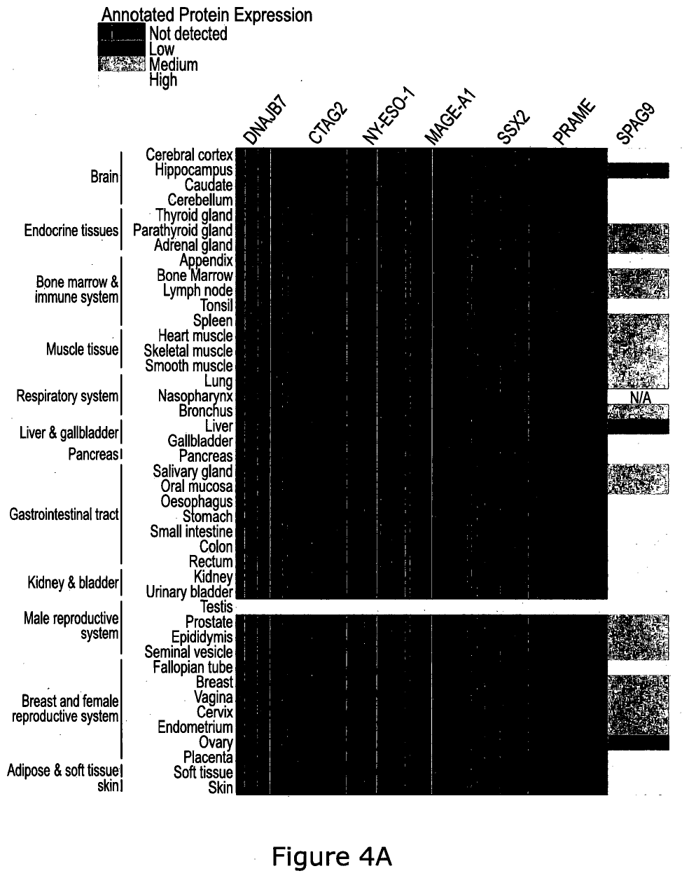

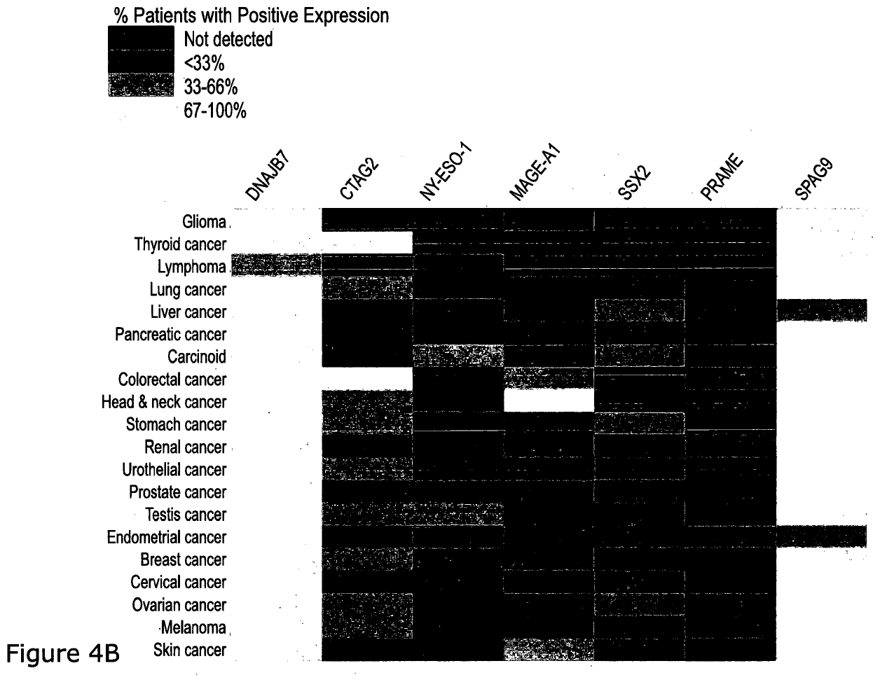

FIG. 4. Expression profile of DNAJB7 in comparison to pre-defined cancer-testis antigens. Utilising the Protein Atlas, the protein expression of DNAJB7 was evaluated alongside other well-defined cancer-testis antigens in a range of healthy tissues (A) and tumour types by location (B).

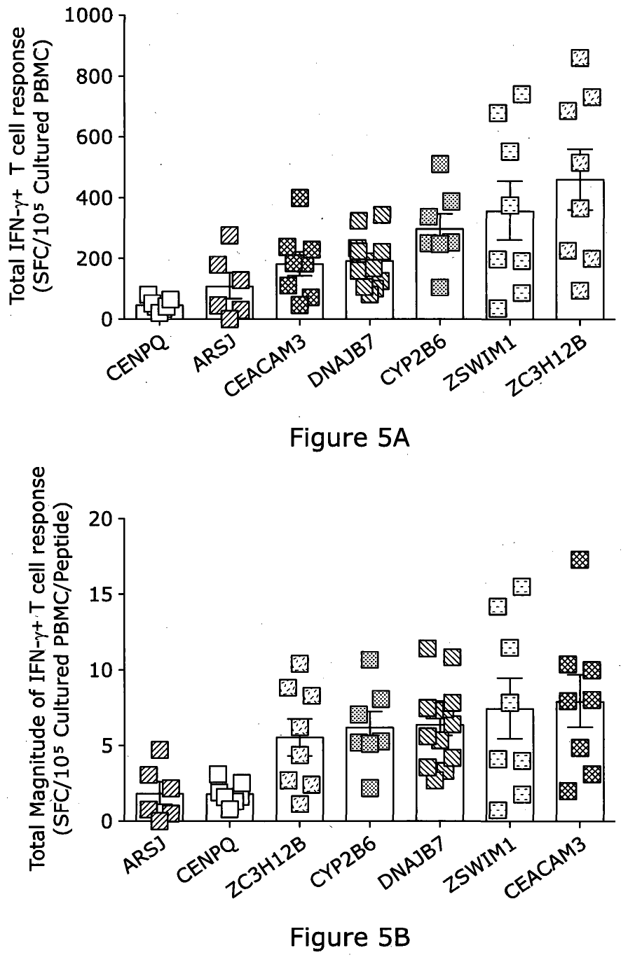

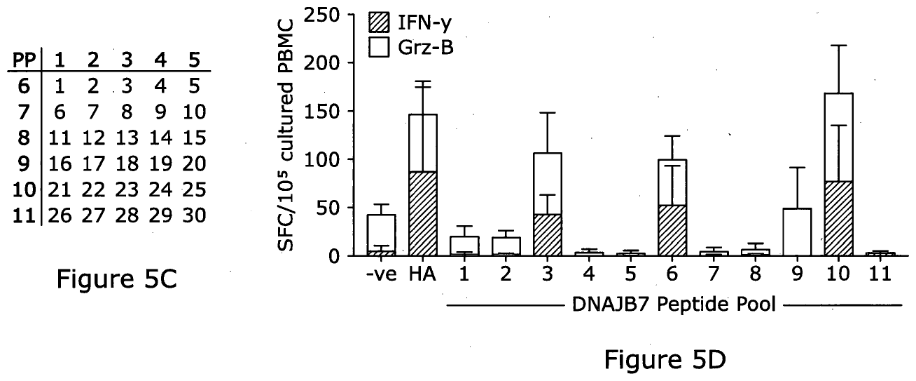

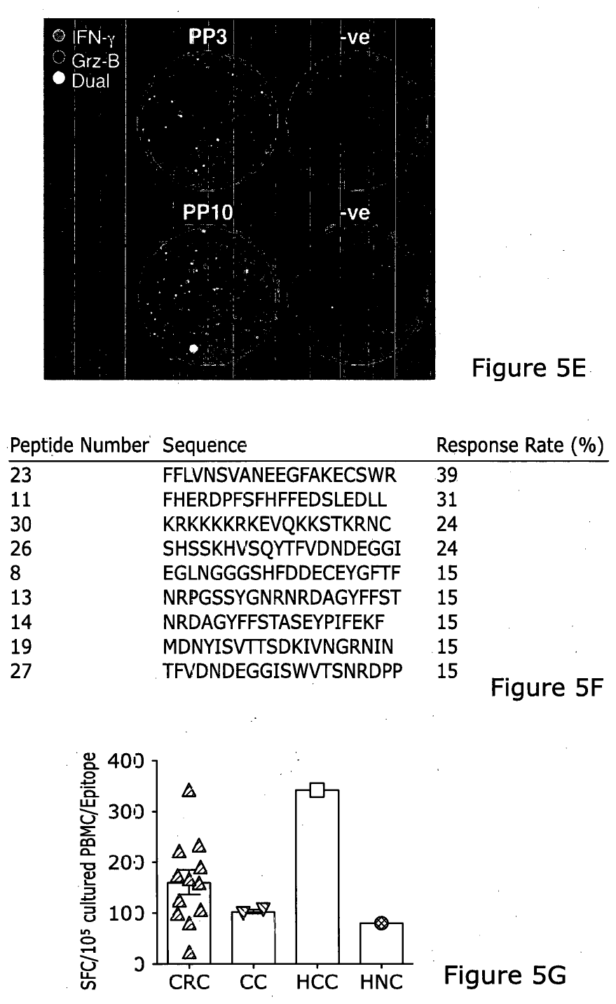

FIG. 5. Immunogenicity of candidate TAAs. T cell responses to peptide pools spanning the entire protein sequence of each candidate TAA was assessed by cultured IFN-y FluoroSpot (see Tables 4 for peptide sequences). The total number of IFN-y+spot-forming cells per 10⁵ cultured PBMC to each positive peptide pool (A) and the overall magnitude relative to the number of peptides in the protein (B) was assessed amongst CRC patients. (C) The DNAJB7 protein was divided into 30 20mers overlapping by 10 (see Table 2 for details), which were then placed into 2 of 11 peptide pools as shown. (D) Representative example of the identification of immunogenic DNAJB7 peptides in a CRC patient by IFN-γ/Granzyme B FluoroSpot. (E) This patient mounted both an IFN-γ and Granzyme B response to peptide pools 3, 6 and 10, indicative of response to peptides 3 and 23, example well images shown. (F) The response rates of peptide sequences above 10% reveals peptide 23 to be the most immunogenic amongst all donors tested. (G) Patients with all cancers tested thus far appear to mount anti-DNAJB7 T cell responses (CRC—colorectal cancer; CC—cholangiocarcinoma; HCC—hepatocellular carcinoma; HNC—head and neck squamous cell carcinoma).

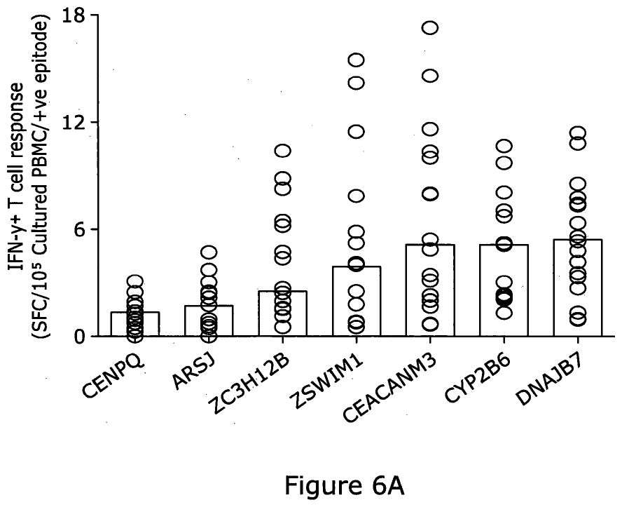

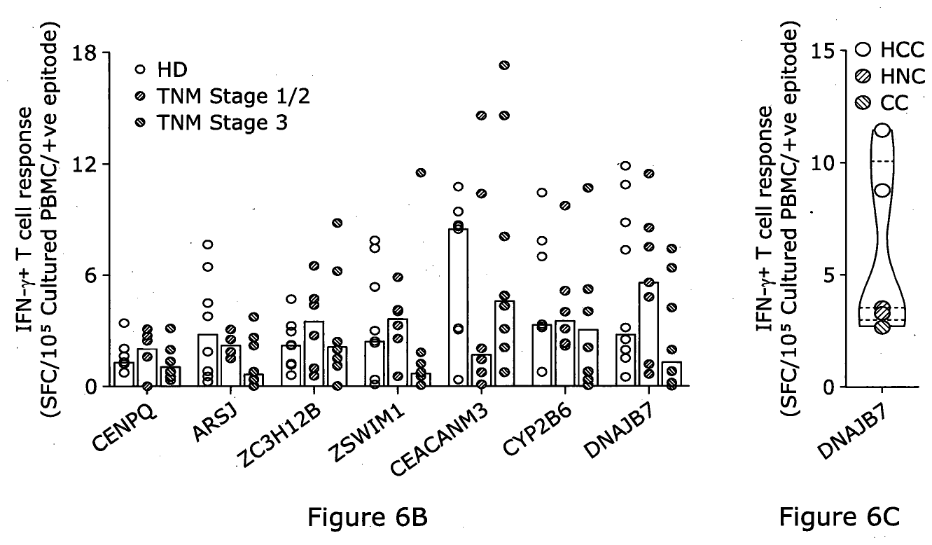

FIG. 6. Immunogenicity of candidate TAAs. T cell responses to peptide pools spanning the entire protein sequence of each candidate TAA were assessed by cultured IFN-γ ELISpot. The total number of IFN-γ⁺ spot-forming cells (SFC) per 10⁵ cultured PBMC relative to the number of peptides in the protein was assessed and ranked by median response amongst all donors tested (A) and then subdivided by CRC patients (blue circles TNM Stage 1/2; n=6, black circles TNM Stage 3; n=8) and healthy donors (‘HD’, white circles; n=10) (B). (C) Patients with other gastrointestinal cancers were tested for their ability to mount anti-DNAJB7 T cell responses (CC—cholangiocarcinoma; HCC—hepatocellular carcinoma; HNC—head and neck squamous cell carcinoma).

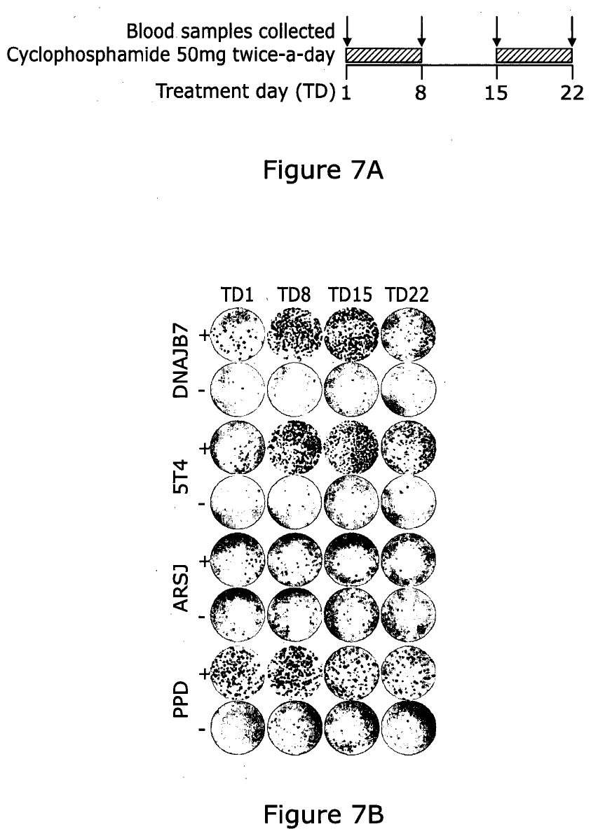

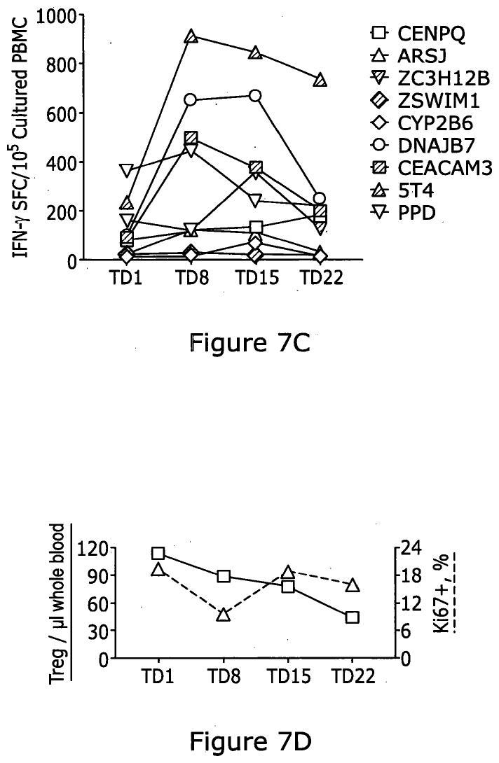

FIG. 7. T_{H}1 responses to certain novel TAAs are unmasked by regulatory T cell depletion in colorectal cancer (CRC). (A) A post-colectomy CRC patient received low-dose, metronomic cyclophosphamide on treatment days 1-8 and 15-22, with blood samples collected weekly throughout treatment. T cell responses to peptide pools spanning the entire protein sequence of each candidate TAA were assessed by cultured IFN-γ ELISpot at each timepoint; example images of IFN-γ ELISpot wells are shown (B). (C) The total number of IFN-γ⁺ spot-forming cells (SFC) per 10⁵ cultured PBMC (mean of duplicate wells) were calculated for each TAA. (D) CD3⁺CD4⁺CD25^{hi}Foxp3⁺ regulatory T cell numbers and %Ki67⁺ Tregs were measured by flow cytometry during cyclophosphamide treatment.

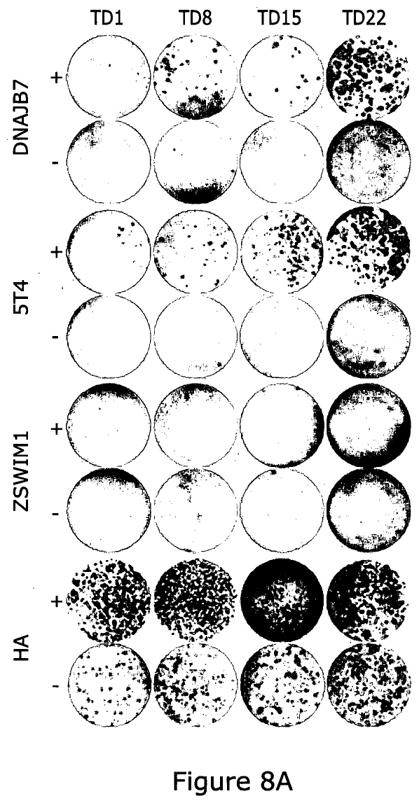

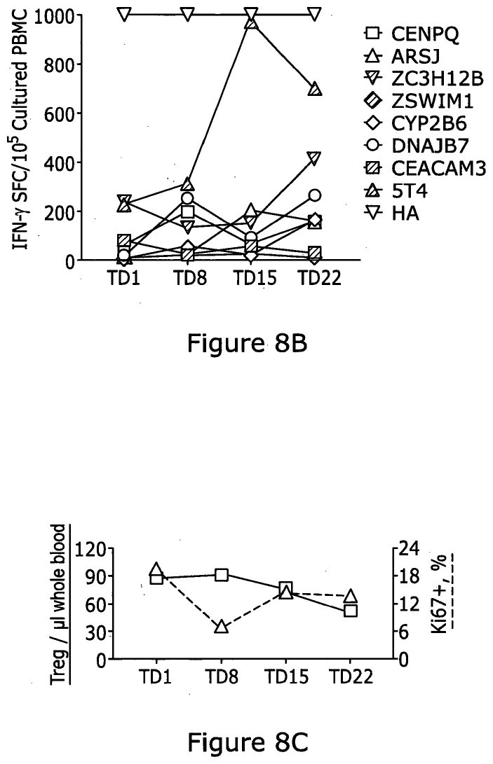

FIG. 8. T_{H}1 responses to certain novel TAAs are unmasked by regulatory T cell depletion in hepatocellular cancer (HCC). An HCC patient received low-dose, metronomic cyclophosphamide on treatment days 1-8 and 15-22, with blood samples collected weekly throughout treatment. T cell responses to peptide pools spanning the entire protein sequence of each candidate TAA were assessed by cultured IFN-γ ELISpot at each timepoint; example images of IFN-γ ELISpot wells are shown (A). (B) The total number of IFN-γ⁺ spot-forming cells (SFC) per 10⁵ cultured PBMC (mean of duplicate wells) were calculated for each TAA. (C) CD3⁺CD4⁺CD25^{hi}Foxp3⁺ regulatory T cell numbers and %Ki67⁺ Tregs were measured by flow cytometry during cyclophosphamide treatment.

Table 1. Details of all 23 genes identified by final analysis. ‘*’ highlights those taken forward, ‘**’ denotes those which were not protein coding.

Table 2. Amino acid sequence of DNAJB7 (SEQ ID NO: 31; accession number NP_660157.1)

Table 3. Nucleic acid sequence of DNAJB7 (SEQ ID NO: 32; accession number NM_145174.1)

Table 4. DNAJB7 Peptide Sequences.

### DETAILED DESCRIPTION

Methods and Materials

Patient Treatment Schedule Orally administered 50 mg cyclophosphamide was taken twice-a-day on treatment days 1-7 and 15-21; no cyclophosphamide was taken on treatment days 8-14 or 22-106, or until patient relapsed. Peripheral blood samples (40 mL) were taken at regular intervals during therapy.

Excision of Colonic and Tumour Tissue

Colorectal tumour and paired background (unaffected) colon specimens were obtained from three patients undergoing primary tumour resection for colorectal adenocarcinoma at the University Hospital of Wales, Cardiff. Autologous colon samples were cut from macroscopically normal sections of the excised tissue, both “near” (within 2 cm) and “far” (at least 10 cm) from the tumour site (FIG. 1A). All fresh tumour samples were derived from the luminal aspect of the specimen, so as not to interfere with the deep part of the tumour required for histopathological staging. Informed consent was obtained from all participants. The Wales Research Ethics Committee granted ethical approval for this study.

Purification of Tissue Samples

Background colon and tumour specimens were transported and washed in extraction medium comprising RPMI supplemented with penicillin, streptomycin and L-glutamine (Gibco), 2% human AB serum (Welsh Blood Service), 20 μg/ml gentamicin (ThermoFisher) and 2 μg/ml Fungizone (ThermoFisher). Within 30 minutes of resection from a patient, samples were minced with blades in a Petri dish and forced through 70 μm cell strainers to collect a single cell suspension. In no instances were collagenase or DNase treatments used. Dissociated cell preparations of tumour, near and far healthy colonic tissue were initially stained with the amine-reactive viability dye Live/Dead fixable Aqua (ThermoFisher) followed by surface marker staining with CD3-APC (BioLegend) and EpCAM-PE (Miltenyi Biotec) antibodies. EpCAM was chosen as it would enable isolation of epithelial populations over stromal tissue and immune populations (Martowicz et al., 2016; Schnell et al., 2013), with CD3 used to ensure T cell populations were not included in downstream analysis. Samples were resuspended in FACS buffer (PBS, 2% BSA) prior to sorting into Live/Dead⁻EpCAM⁺CD3⁻ populations on a FACS Aria III (BD); gating strategy is shown (FIG. 1C). Tumour tissue also stained with CD3 and EpCAM antibodies was additionally passed through the cell sorter without gating, and used as an unsorted control for RNA-seq. All samples were sorted directly into RLT buffer (Qiagen) with beta mercapto-ethanol (Sigma Aldrich) and frozen at −80° C. Frozen samples were thawed and RNA isolated using a RNA easy micro isolation kit (Qiagen).

RNA Sequencing

Library preparation and RNA sequencing was carried out by VGTI-FL (Florida, USA). Purified RNA was used to make libraries using a TruSeq kit (Illumina). Libraries were sequenced to a depth of 37-63M read pairs on an Illumina HiSeq platform. Paired end reads were processed on a Cardiff University pipeline, trimmed, mapped and quality control analysis performed. Reads were trimmed with Trimmomatic (Bolger et al., 2014) and assessed for quality using FastQC (https://www.bioinformatics.babraham.ac.uk/projects/fastqc/) using the default parameters. Reads were mapped to Ensembl human genome build GRCh38.89 downloaded from the Ensembl FTP site (http:/www.ensembl.org./info/data/ftp/index.html/) using STAR (Dobin et al., 2013).

Differential Expression Analysis

Aligned reads were normalised using DESEQ2 in R (Love et al., 2014). Differentially expressed genes were identified between purified tumour samples, purified near, and purified far epithelium. Differential expression analysis was carried out using DESEQ2 between sample types for all donors in a paired analysis. Comparisons of tumour and near or far tissue were carried out, with reads standardised for comparison using fragments per kilobase of transcript per million mapped reads (FPKM values). For the three-donor expression analysis, genes with a log 2-fold change greater than 3.5, FPKM values in healthy tissue less than 3.5, and FPKM values in tumour greater than 4.0 in any two of three donors as well as an adjusted P-value less than 0.05 in three donors were taken forward for further analysis.

The analysis was expanded to genes which were significantly differentially expressed in separate comparisons for data in two of the three donors (this helped identify genes which were not expressed in one donor but were highly expressed in the remaining two donors). Higher expression cut off values were used with FPKM greater than 5.0 in both donor's tumour tissue, and less than 1.0 in healthy tissues, with a log 2-fold change greater than 6, and adjusted p-value of less than 0.05. Gene lists arising from these comparisons were taken forward for further analysis. Inspection of reads mapped was carried out using integrative genomic viewer software (Broad institute).

PBMC Culture

Blood samples were collected in 10 ml lithium heparin tubes (BD Biosciences) no more than 7 days prior to surgery. Peripheral blood mononuclear cells (PBMCs) were isolated by centrifugation of heparinised blood over Lymphoprep (Axis-Shield). Cells were then washed and re-suspended in CTL Test Plus media (CTL Europe), L-glutamine and penicillin/streptomycin. PBMC were plated in 96-well plates (Nunc) and cultured in triplicate wells with specific antigens for 14 days, supplemented with fresh media containing 20 IU/ml IL-2 on days 3, 7 and 10.

ELISpot Assays

IFN-γ ELISpot assays were performed to assess for novel tumor antigen-specific T cell responses, as previously described (Scurr et al. 2017). Briefly, PVDF 96-well filtration plates were coated with 50 μI IFN-γ antibody (Mabtech). Cells were washed, plated, and stimulated with 5 μg/mlantigen in duplicate wells. Plates were incubated at 37° C., 5% CO2 for 24 hours before removing cells and developing spots. Spot-forming cells (SFC), i.e. IFN-γ-producing T cells, were enumerated using Smart Count settings on an automated ELISpot plate reader (ImmunoSpot S6 Ultra; CTL Europe GmbH). Positive responses were identified as having at least 20 SFC/10⁵ cultured PBMCs, and at least double that of the negative (no antigen) control. Wells with spot counts >1000 were deemed too numerous to count and capped at this level.

FluoroSpot Assays

IFN-γ/Granzyme B FluoroSpot assays were performed to assess for novel tumour antigen-specific T cell responses, as previously described (Scurr et al. 2017). Briefly, PVDF 96-well filtration plates designed for low autofluorescence (IPFL; Millipore) were used for all FluoroSpot assays. Antibodies to IFN-γ and Granzyme B, and fluorescence enhancer kits were obtained from Mabtech. All antibody incubations were with 50 μl/well. Cells were then washed, plated, and stimulated with 5 μg/mL antigen in duplicate wells. Plates were incubated at 37° C., 5% CO₂ for 24 hours. Cytokine-producing T cells were enumerated using Smart Count settings on an automated FluoroSpot plate reader (ImmunoSpot S6 Ultra; CTL Europe GmbH), allowing for an assessment of single and dual cytokine-producing cells. Positive responses were identified as having at least 5 SFC/10⁵ PBMCs, and at least double that of the negative (no antigen) control.

Antigens

20mer peptides overlapping by 10 amino acids, covering the entire protein sequence of each identified TAA, were synthesised by Fmoc chemistry to >95% purity (GL Biochem, Shanghai, China), and divided into pools, as shown (Table 4). The recall antigens tuberculin purified protein derivative (PPD; Statens Serum Institut) and hemagglutinin (HA; gift from Dr. John Skehel, National Institute of Medical Research, London, United Kingdom), and the T cell mitogen PHA (Sigma) were used as positive controls. All antigens were used at a final concentration of 5 μg/ml.

Immunohistochemistry

The identified TAAs from this study were evaluated for protein expression characteristics on healthy tissue and a range of tumour samples by utilising the Human Protein Atlas resource (Uhlén M et al. 2015).

Results

Purification of Samples Prior to RNA-Seq Analysis Provided Enhanced Resolution Of Differentially Expressed Genes

RNA-seq datasets were comparable following several normalisation procedures. Differential expression comparisons were run using DESEQ2 of healthy tissues (“near” and “far”) against purified tumour tissue in all three patients, and then separate analyses for each combination of two patients. An additional comparison of non-purified tumour tissue against healthy tissues was run to investigate the impact of EpCAM sorting. To find relevant genes that could be targeted by immunotherapy, we applied criteria that specified low levels of expression in healthy tissue combined with high expression in tumour tissue (based on FPKM and log 2-fold change). Only genes assigned an adjusted P-value<0.05 were taken forward for further analysis.

Initial gene lists gave 83 significant genes showing differential expression between tumour and far colon tissue, while 92 genes between tumour and near colon tissue. Cross referencing of these gene lists resulted in 5 genes that satisfied significant criteria in both comparisons (including 4 of those taken forward; ARSJ, CENPQ, ZC3H12B and CEACAM3). To expand our analysis, we looked at DEGs which were significantly expressed in tumour tissue of two of three patients to a higher level (increased expression cut-offs and lower threshold of healthy tissue expression). These gene lists were combined with three donor lists, and then near and far tissue cross referenced (FIG. 2A). This gave an initial set of 54 genes which were cut to 23 based on levels of expression in healthy tissue of all three donors. Of these 23 genes, 18 were protein coding. We inspected these 18 genes and selected those which were most suitable for further analysis, eliminating those involved in the central nervous system, or which exhibited an inconsistent expression or read mapping profile in three donors gauged by visual curation of mapped reads in IGV.

The final genes selected were DNAJB7, CENPQ, ZC3H12B, ZSWIM1, CEACAM3, ARSJ and CYP2B6, based on their ideal expression profile for therapeutic exploitation (FIG. 2B). Inspection of relevant expression profiles in RNA-seq data from non-purified tumour tissue exemplifies how none of these genes were detected in non-purified tissue of all donors, with expression levels much lower than purified tissue. Those that were detected in non-purified tissue of two donors did not come up as significant in differential expression analysis by DESEQ2, again emphasising the power of purification prior to RNA-seq analysis.

Analysis of Protein Expression Across Multiple Healthy Tissues Highlights DNAJB7 as a Cancer-Testis Antigen and a Suitable Target for Immunotherapy

The protein expression level of each candidate TAA was evaluated using immunohistochemistry data publicly available in the Human Protein Atlas. Whilst each candidate exhibited significant upregulation on tumour tissue over healthy tissue, although more limited for ARSJ, DNAJB7 was unexpectedly identified as a novel cancer-testis antigen given its complete lack of expression on any healthy tissue bar the testis, an immune-privileged site (FIG. 3A-C). Furthermore, DNAJB7, a protein belonging to the evolutionarily conserved DNAJ heat shock family, was expressed on a very wide range of solid tumours, in particular on tumours of the gastrointestinal tract and accessory organs of digestion, including colorectal cancer and pancreatic ductal adenocarcinoma (FIG. 3B; example immunohistochemistry data also included FIG. 3C).

The expression profile of DNAJB7 was compared to six other well-defined cancer-testis antigens, including NY-ESO-1, MAGE-A1 and SSX2. High protein expression of all these antigens was confirmed to be confined to the testis, apart from SPAG9 (FIG. 4A). In comparison to the other cancer-testis antigens, DNAJB7 was expressed on the greatest range of tumour types, with more than 67% of all patients tested exhibiting positive (low, medium or high) protein expression on their tumour, except for lymphoma (FIG. 4B). This remarkable protein expression profile sets DNAJB7 aside as an excellent target for broadly applicable cancer immunotherapy.

Analysis of Candidate TAA T_{H}1 Responses Reveal DNAJB7 to be Immunogenic

Following identification of relevant genes and confirmed protein expression, we assessed their immunogenicity using over lapping peptide pools and culture with PBMC of CRC patients and healthy donors. Analysis of cultured PBMC by IFN-γ/Granzyme-B FluoroSpot determined five of the seven proteins to demonstrate immunogenicity in the majority of donors (FIGS. 5A-B and 6A-B). As the size of peptide libraries was highly variable for each protein, we standardised the immunogenicity relevant to the number of peptides in each pool (FIGS. 5B and 6B). This analysis revealed CYP2B6, DNAJB7, and CEACAM3 to be comparably immunogenic across multiple individuals without stratification by HLA-type. Conversely, CENPQ and ARSJ were poorly immunogenic in most donors tested. DNAJB7 was also found to be immunogenic in patients with other tumor types, including hepatocellular carcinoma, cholangiocarcinoma and the non-gastrointestinal head and neck squamous cell cancer (FIGS. 5G and 6C).

Furthermore, our peptide pool design, as described before, allowed us to interrogate immunogenicity based on a matrix format to determine the peptides responsible for the positive T cell responses (example for DNAJB7, FIGS. 5C-F and 6). This type of analysis is important for isolation of T_{H}1 stimulating regions of the TAA, which may be incorporated in vaccines based on immunogenic components of multiple antigens important in CRC, as well as being regions that can be targeted by epitope-based modifications and strategies for enhancement of the immune response. A representative example of one CRC patient revealed positive IFN-γ and granzyme B responses to DNAJB7 peptide pools 3, 6 and 10, indicative of T cell responses to epitopes contained within peptides 3 and 23 (specifically peptides 11, 30, 26, 8, 13, 14, 19 and 27; FIG. 5D and E). Notably, these immunogenic fragments encompass significant portions of the full length DNAJB7 peptide, spanning from close to the C-terminus to the N-terminus, which advantageously demonstrate fragments spanning the entire length of the protein can elicit a response. Peptide 23 was the most immunogenic region of the DNAJB7 protein, with responses discovered in 39% of CRC patient and healthy control donors tested (FIG. 5F). DNAJB7 was tested in hepatocellular carcinoma, cholangioma, and head and neck cancer, and was also found to be immunogenic in these patients (FIGS. 5G and 6C).

Anti-DNAJB7 T_{H}1 Responses are Induced During Cyclophosphamide Treatment

We have previously demonstrated that anti-tumor T_{H}1 effector responses are controlled by regulatory T cells (Tregs), and that targeting these Tregs either by depletion in vitro, or inhibition/depletion in vivo with low dose cyclophosphamide, increases the anti-tumor (5T4) immune response (Scurr et al. 2017). We sought to assess whether T cell responses were induced to the novel tumor antigens in a colorectal cancer (CRC) patient (FIG. 7) and a hepatocellular (HCC) patient (FIG. 8) receiving short-term metronomic cyclophosphamide. Anti-5T4 T_{H}1 responses increased by >4-fold in both patients, an effect previously identified as associating with improved survival outcomes (Scurr et al. 2017); intriguingly, anti-DNAJB7 T_{H}1 responses also mirrored this treatment response profile in both instances, whereas no responses were induced to ARSJ, CENPQ, ZSWIM1 and CYP2B6 (FIG. 7B and C, and FIG. 8A and B). This could suggest that responses to DNAJB7 and CEACAM3 are suppressed in CRC and HCC, given that responses were unmasked by efficient regulatory T cell depletion (FIG. 7D and FIG. 8C).

### SUMMARY

Here, a panel of broadly expressed, novel TAAs were identified (CENPQ, CEACAM3, CYP2B6, DNAJB7, ZC3H12B, ZSWIM1, ARSJ) by performing RNA sequencing of highly purified EpCAM+colorectal tumour cells in comparison to patient-matched EpCAM+colonic epithelial cells, analysing for the most differentially expressed genes. Tumour cell purification was necessary to reveal the genes, demonstrating how prior methods that sequence whole tumour fractions (i.e. inclusive of dead cells, stromal cells, immune cells and other tumour infiltrating cells) for antigen identification are flawed. Protein expression of the candidate TAAs was confirmed by immunohistochemistry, and pre-existing T cell immunogenicity towards these antigens tested by IFN-γ/Granzyme-B FluoroSpot. Of these, DNAJB7 (DnaJ heat shock protein family member B7), was identified here as a novel cancer-testis antigen, given its exceptionally restricted expression to cells of the testis and a wide variety of tumours, including glioma, breast, melanoma, pancreatic, liver, colorectal and renal cancers, was highly immunogenic recognized by effector and memory T cells lending itself lends to use a vaccine candidate in the treatment of a variety of cancers.

TABLE 1

| Ensembl Gene ID | Gene Name | Description |
|---|---|---|
| ENSG00000031691 | CENPQ * | centromere protein Q [Source: HGNC Symbol; Acc: HGNC: 21347] |
| ENSG00000073464 | CLCN4 | chloride voltage-gated channel 4 [Source: HGNC Symbol; Acc: HGNC: 2022] |
| ENSG00000079482 | OPHN1 | oligophrenin 1 [Source: HGNC Symbol; Acc: HGNC: 8148] |
| ENSG00000102053 | ZC3H12B * | zinc finger CCCH-type containing 12B [Source: HGNC Symbol; Acc: HGNC: 17407] |
| ENSG00000103021 | CCDC113 | coiled-coil domain containing 113 [Source: HGNC Symbol; Acc: HGNC: 25002] |
| ENSG00000103184 | SEC14L5 | SEC14 like lipid binding 5 [Source: HGNC Symbol; Acc: HGNC: 29032] |
| ENSG00000151632 | AKR1C2 | aldo-keto reductase family 1 member C2 [Source: HGNC Symbol; Acc: HGNC: 385] |
| ENSG00000159263 | SIM2 | single-minded family bHLH transcription factor 2 [Source: HGNC Symbol; Acc: HGNC: 10883] |
| ENSG00000162929 | KIAA1841 | KIAA1841 [Source: HGNC Symbol; Acc: HGNC: 29387] |
| ENSG00000166529 | ZSCAN21 | zinc finger and SCAN domain containing 21 [Source: HGNC Symbol; Acc: HGNC: 13104] |
| ENSG00000166762 | CATSPER2 | cation channel sperm associated 2 [Source: HGNC Symbol; Acc: HGNC: 18810] |
| ENSG00000168612 | ZSWIM1 * | zinc finger SWIM-type containing 1 [Source: HGNC Symbol; Acc: HGNC: 16155] |
| ENSG00000170956 | CEACAM3 * | carcinoembryonic antigen related cell adhesion molecule 3 [Source: HGNC |
| | | Symbol; Acc: HGNC: 1815] |
| ENSG00000172404 | DNAJB7 * | DnaJ heat shock protein family (Hsp40) member B7 [Source: HGNC Symbol; Acc: HGNC: 24986] |
| ENSG00000180801 | ARSJ * | arylsulfatase family member J [Source: HGNC Symbol; Acc: HGNC: 26286] |
| ENSG00000181638 | ZFP41 | ZFP41 zinc finger protein [Source: HGNC Symbol; Acc: HGNC: 26786] |
| ENSG00000197408 | CYP2B6 * | cytochrome P450 family 2 subfamily B member 6 [Source: HGNC Symbol; Acc: HGNC: 2615] |
| ENSG00000214376 | VSTM5 | V-set and transmembrane domain containing 5 [Source: HGNC Symbol; Acc: HGNC: 34443] |
| ENSG00000254343 | AC091563.1 ** | |
| ENSG00000255104 | AC005324.4 ** | |
| ENSG00000267506 | AC021683.2 ** | |
| ENSG00000272791 | AC073389.3 ** | |
| ENSG00000283799 | MIR1182 ** | microRNA 1182 [Source: HGNC Symbol; Acc: HGNC: 35263] |

TABLE 2

| | |
|---|---|
| 1 | mvdyyevlgl qryaspedik kayhkvalkw |
| | hpdknpenke eaerkfkeva eayevlsnde |
| 61 | krdiydkygt eglngggshf ddeceygfff |
| | hkpddvfkei fherdpfsfh ffedsledll |
| 121 | nrpgssygnr nrdagyffst aseypifekf |
| | ssydtgytsq gslgheglts fsslafdnsg |
| 181 | mdnyisvtts dkivngrnin tkkiiesdqe |
| | reaedngelt fflvnsvane egfakecswr |
| 241 | tqsfnnyspn shsskhvsqy tfvdndeggi |
| | swvtsnrdpp ifsagvkegg krkkkkrkev |
| 301 | qkkstkrnc |

TABLE 3

| | |
|---|---|
| 1 | taacattgtc tttaacataa aagtgtacct atttgcagtc actacctcta tcaccaccac |
| 61 | cagcagagcc tgagctgagg aaaccacggt tctcaatacc cagcacaccc acttccaact |
| 121 | atctgttaaa acatggtgga ttactatgaa gttctaggac tgcaaagata tgcttcacct |
| 181 | gaggacatta aaaaagctta tcataaagtg gcacttaaat ggcaccctga taaaaatcca |
| 241 | gaaaataaag aagaagcaga gagaaaattc aaagaagtag ctgaggcata cgaggtatta |
| 301 | tcaaatgatg agaaacggga catttatgat aaatatggca cagaaggatt aaacggaggt |
| 361 | ggaagtcatt ttgatgatga atgtgagtac ggcttcacat tccataagcc agatgatgtt |
| 421 | tttaaagaaa tttttcatga aagggatcca ttttcttttc acttctttga agactcgctt |
| 481 | gaggacctgt taaatcgtcc aggaagctcc tatggaaaca gaaacagaga tgcaggatac |
| 541 | tttttctcca ctgccagtga atatccaatt tttgagaaat tttcttcata tgatacagga |
| 601 | tatacatcac agggttcatt ggggcatgaa ggccttactt ctttctcttc cctggctttt |
| 661 | gataatagtg ggatggacaa ctacatatct gttacaactt cagacaaaat cgttaatggc |
| 721 | agaaatatta atacaaagaa aattattgaa agtgatcaag aaagagaagc tgaagataat |
| 781 | ggagagttga cattttttct tgtaaatagt gtggccaatg aagagggctt tgcaaaagaa |
| 841 | tgcagctgga gaacacagtc attcaacaac tattcaccaa attctcacag ctccaaacat |
| 901 | gtatctcaat atactttcgt ggacaatgat gagggaggta tatcttgggt taccagcaac |
| 961 | agagatcccc ctattttctc agcaggagtc aaagagggtg gtaagaggaa aaaaaagaag |
| 1021 | cgtaaagagg tgcaaaagaa gtctaccaaa aggaattgtt aaattgactc ttcaaatata |
| 1081 | taacatttga acacaattgt gtgtgttttg gttaatcaca aattttgtag ataacactta |
| 1141 | atactatact aagagctttt caacactttt agcaggattg tggacatttg gttagtagtt |
| 1201 | tttttgaatg ggtatgtcag aaaaggatga gtttgtggtg acagttggtg ctaataagaa |
| 1261 | tttgcctggg cagtatagtg agatcttttc tctacaaaaa atttaaaaat tagccaagtg |
| 1321 | tggtggtgtg cacctgtagt cccagctact cgggaagctg gtagaaagac tgcttgagcc |
| 1381 | tggaagacat aggttgcagt gtaatgggat cacgccactg cactccagct tgggtgacaa |
| 1441 | agtaagaccc tgtcttaaaa aaaaaaaaaa ggaatttgga aaactgcaat gagtttttta |
| 1501 | cttttgaatt tttctttagt agagtttata gcctattttt cttttttcta attaaaacca |
| 1561 | ctatacaaac ttacgaaaca tgcatagcat tagaagacaa gaatgtatca agacacacag |
| 1621 | gttcagacta cacaattatt catgaaatgg ctgttttcca atggaggaac agcatttgat |
| 1681 | gcattataac ttaaaggtac tacaaattaa aaattaaagt ataaagtatt ttaagttaga |
| 1741 | aatagactta gctgggtgca gtggctcaca cctgtaatcc tagcatcttg ggaggcccag |
| 1801 | gcaggagggt tgaagtcaga atttcacgac agtccgggta acataatgag acctgtcctc |
| 1861 | tatgaaaaaa cttttttaaa aatttagctg ggtgtggtgg tacgtgcctg taggcctagc |
| 1921 | tacttaggag gctgaggtgg gaggattgct tgagcccagg agtttgaggt tgtagggagc |
| 1981 | tatgatcttg ccattgcact ccagctgaga gtgtgaccct gtctctaaag aaaaactata |
| 2041 | gaaaatttaa aaaattaaaa aacgggtaac atggtaaatt ttatgtagta tattttacca |
| 2101 | catttttttt aaaggccagt cttatcctga attcctcaga gcagtctgta tggtaggcca |
| 2161 | tcagtgaatc actccagaca ggaaattttc tatttttgtt tagagtttgt acacaaaatc |
| 2221 | tacaaataca ttactctaaa atagcaccta tagccatttt aatttagtca tgttgggaga |
| 2281 | aaaatagatg ggttacaaat acatcagata tcgagcaaat ggtttgagtt acatctagca |
| 2341 | gcactaaaac ttattcaact ttggccaaat acaaatgcga aattagaaaa aaattataat |
| 2401 | atctgctatt tttaaaagac agctatagtc aagagcttgt gttttgtatc ggtatttgat |
| 2461 | ttttagtggg cagatgggag aaggaaaggt tatttattct atagaagaga ctgcggacaa |
| 2521 | ttagtattca aagtttattt tcagaaaata aaataataaa agcccagggt attttaaa |

TABLE 4
29 20mers, 1 19mer, overlapping
by 10aa sequences.
Peptide Pool 1 = Peptides 1-15;
Peptide Pool 2 = Peptides 16-30.

| | |
|---|---|
| 1) | MVDYYEVLGLQRYASPEDIK (SEQ ID NO: 1); |
| 2) | QRYASPEDIKKAYHKVALKW (SEQ ID NO: 2); |
| 3) | KAYHKVALKWHPDKNPENKE (SEQ ID NO: 3); |
| 4) | HPDKNPENKEEAERKFKEVA (SEQ ID NO: 4); |
| 5) | EAERKFKEVAEAYEVLSNDE (SEQ ID NO: 5); |
| 6) | EAYEVLSNDEKRDIYDKYGT (SEQ ID NO: 6); |
| 7) | KRDIYDKYGTEGLNGGGSHF (SEQ ID NO: 7); |
| 8) | EGLNGGGSHFDDECEYGETF (SEQ ID NO: 8); |
| 9) | DDECEYGETFHKPDDVEKEI (SEQ ID NO: 9); |
| 10) | HKPDDVFKEIFHERDPFSFH (SEQ ID NO: 10); |
| 11) | FHERDPFSFHFFEDSLEDLL (SEQ ID NO: 11); |
| 12) | FFEDSLEDLLNRPGSSYGNR (SEQ ID NO: 12); |
| 13) | NRPGSSYGNRNRDAGYFFST (SEQ ID NO: 13); |
| 14) | NRDAGYFFSTASEYPIFEKE (SEQ ID NO: 14); |
| 15) | ASEYPIFEKFSSYDTGYTSQ (SEQ ID NO: 15); |
| 16) | SSYDTGYTSQGSLGHEGLTS (SEQ ID NO: 16); |
| 17) | SLGHEGLTSFSSLAFDNSG (SEQ ID NO: 17); |
| 18) | FSSLAFDNSGMDNYISVTTS (SEQ ID NO: 18); |
| 19) | MDNYISVTTSDKIVNGRNIN (SEQ ID NO: 19); |
| 20) | DKIVNGRNINTKKIIESDQE (SEQ ID NO: 20); |
| 21) | TKKIIESDQEREAEDNGELT (SEQ ID NO: 21); |
| 22) | REAEDNGELTFFLVNSVANE (SEQ ID NO: 22); |
| 23) | FFLVNSVANEEGFAKECSWR (SEQ ID NO: 23); |
| 24) | EGFAKECSWRTQSENNYSPN (SEQ ID NO: 24); |
| 25) | TQSENNYSPNSHSSKHVSQY (SEQ ID NO: 25); |
| 26) | SHSSKHVSQYTEVDNDEGGI (SEQ ID NO: 26); |
| 27) | TFVDNDEGGISWVTSNRDPP (SEQ ID NO: 27); |
| 28) | SWVTSNRDPPIFSAGVKEGG (SEQ ID NO: 28); |
| 29) | IFSAGVKEGGKRKKKKRKEV (SEQ ID NO: 29); |
| 30) | KRKKKKRKEVQKKSTKRNC (SEQ ID NO: 30); |

### REFERENCES

Ausubel F M et al, Current Protocols in Molecular Biology, John Wiley & Sons, Inc. (1994).

Bolger, A.M., Lohse, M., and Usadel, B. (2014). Trimmomatic: a flexible trimmer for IIlumina sequence data. Bioinformatics 30, 2114-20

Dobin, A., Davis, C.A., Schlesinger, F., Drenkow, J., Zaleski, C., Jha, S., Batut, P., Chaisson, M., and Gingeras, T. R. (2013). STAR: ultrafast universal RNA-seq aligner. Bioinformatics 29, 15-21.

Grosveld et al., Cell (1987), 51: 975-985

Latchman, David S, Eukaryotic Transcription Factors, by Academic Press Ltd, San Diego.

Love, M. I., Huber, W., and Anders, S. (2014). Moderated estimation of fold change and dispersion for RNA-seq data with DESeq2. Genome Biol. 15, 550.

Marston, F (1987) DNA Cloning Techniques: A Practical Approach Vol III IRL Press, Oxford UK; DNA Cloning.

Martowicz, A., Seeber, A., and Untergasser, G. (2016). The role of EpCAM in physiology and pathology of the epithelium. Histol. Histopathol. 31, 349-355.

Sambrook et al (1989) Molecular Cloning: A Laboratory Manual, Cold Spring Harbour Laboratory, Cold Spring Harbour, N.Y. and references therein.

Schnell, U., Cirulli, V., and Giepmans, B. N. G. (2013). EpCAM: Structure and function in health and disease. Biochim. Biophys. Acta-Biomembr. 1828, 1989-2001.

Scurr, M., Pembroke, T., Bloom, A., Roberts, D., Thomson, A., Smart, K., Bridgeman, H., Adams, R., Brewster, A., Jones, R., et al. (2017). Effect of Modified Vaccinia Ankara-5T4 and Low-Dose Cyclophosphamide on Antitumor Immunity in Metastatic Colorectal Cancer. JAMA Oncol. 3, e172579.

Uhlén, M., Fagerberg L., Hallström B.M., Lindskog C., Oksvold P., Mardinoglu A., Sivertsson A., Kampf C., Sjöstedt E., Asplund A., et al. (2015). Proteomics. Tissue-based map of the human proteome. Science. 347, 1260419.

1. An immunogenic agent for use as a cancer vaccine comprising or consisting of DnaJ heat shock protein family (Hsp40) member B7 (termed DNAJB7) or at least one immunogenic fragment thereof.

2. The immunogenic agent wherein DNAJB7 is human DNAJB7.

3. The immunogenic agent according to claim 2 wherein DNAJB7 is represented by the amino acid sequence set forth in SEQ ID NO: 31 or a sequence having at least 85%, 86%, 87%, 88%, 89%, 90%, 91%, 92%, 93%, 94%, 95%, 96%, 97%, 98% or 99% identity therewith.

4. The immunogenic agent according to claim 1, wherein said at least one fragment is 5-30 amino acids in length.

5. The immunogenic agent according to claim 1, wherein said at least one DNAJB7 immunogenic fragment comprises or consists of an amino acid sequence selected from the group of sequence listing consisting of:
a)
(SEQ ID NO: 1)
MVDYYEVLGLQRYASPEDIK;
(SEQ ID NO: 2)
QRYASPEDIKKAYHKVALKW;
(SEQ ID NO: 3)
KAYHKVALKWHPDKNPENKE;
(SEQ ID NO: 4)
HPDKNPENKEEAERKFKEVA;
(SEQ ID NO: 5)
EAERKFKEVAEAYEVLSNDE;
(SEQ ID NO: 6)
EAYEVLSNDEKRDIYDKYGT;
(SEQ ID NO: 7)
KRDIYDKYGTEGLNGGGSHF;
(SEQ ID NO: 8)
EGLNGGGSHFDDECEYGFTF;
(SEQ ID NO: 9)
DDECEYGFTFHKPDDVFKEI;
(SEQ ID NO: 10)
HKPDDVFKEIFHERDPFSFH;
(SEQ ID NO: 11)
FHERDPFSFHFFEDSLEDLL;
(SEQ ID NO: 12)
FFEDSLEDLLNRPGSSYGNR;
(SEQ ID NO: 13)
NRPGSSYGNRNRDAGYFFST;
(SEQ ID NO: 14)
NRDAGYFFSTASEYPIFEKF;
(SEQ ID NO: 15)
ASEYPIFEKFSSYDTGYTSQ;
(SEQ ID NO: 16)
SSYDTGYTSQGSLGHEGLTS;
(SEQ ID NO: 17)
GSLGHEGLTSFSSLAFDNSG;
(SEQ ID NO: 18)
FSSLAFDNSGMDNYISVTTS;
(SEQ ID NO: 19)
MDNYISVTTSDKIVNGRNIN;
(SEQ ID NO: 20)
DKIVNGRNINTKKIIESDQE;
(SEQ ID NO: 21)
TKKIIESDQEREAEDNGELT;
(SEQ ID NO: 22)
REAEDNGELTFFLVNSVANE;
(SEQ ID NO: 23)
FFLVNSVANEEGFAKECSWR;
(SEQ ID NO: 24)
EGFAKECSWRTQSFNNYSPN;
(SEQ ID NO: 25)
TQSFNNYSPNSHSSKHVSQY;
(SEQ ID NO: 26)
SHSSKHVSQYTFVDNDEGGI;
(SEQ ID NO: 27)
TFVDNDEGGISWVTSNRDPP;
(SEQ ID NO: 28)
SWVTSNRDPPIFSAGVKEGG;
(SEQ ID NO: 29)
IFSAGVKEGGKRKKKKRKEV;
and
(SEQ ID NO. 30)
KRKKKKRKEVQKKSTKRNC;
or;
1)) a fragment that has at least 85% sequence identity with any one or more of the sequences in group a) and, in ascending order of preference, at least 86%, 87%, 88%, 89%, 90%, 91%, 92%, 93%, 94%, 95%, 96%, 97%, 98% or 99% sequence identity with any one or more of the sequences in group a); or
c) a fragment that is a variant of any one or more of the sequences in group a) and/or group
b) wherein said variant is modified by the addition, deletion or substitution of one or more amino acid residues in any one or more of the above sequences and/or wherein said variant fragment retains or has enhanced or comparable immunogenicity when compared to the immunogenicity of any one or more of the variants in group a) and/or group b).

6. The immunogenic agent according to claim wherein said at least one DNAJB7 fragment is an amino acid sequence selected from the group consisting of:
a)
(SEQ ID NO: 23)
FFLVNSVANEEGFAKECSWR;
(SEQ ID NO: 11)
FHERDPFSFHFFEDSLEDLL;
(SEQ ID NO: 30)
KRKKKKRKEVQKKSTKRNC;
(SEQ ID NO: 26)
SHSSKHVSQYTFVDNDEGGI;
((SEQ ID NO: 8)
EGLNGGGSHFDDECEYGFTF;
(SEQ ID NO: 13)
NRPGSSYGNRNRDAGYFFST;
(SEQ ID NO: 14)
NRDAGYFFSTASEYPIFEKF;
(SEQ ID NO: 19)
MDNYISVTTSDKIVNGRNIN;
and
(SEQ ID NO: 27)
TFVDNDEGGISWVTSNRDPP;
or
b) a fragment that has at least 85% sequence identity with any one or more of the sequences in group a) and, in ascending order of preference, at least 86%, 87%, 88%, 89%, 90%, 91%, 92%, 93%, 94%, 95%, 96%, 97%, 98% or 99% sequence identity with any one or more of the sequences in group a); or
c) a fragment that is a variant of any one or more of the sequences in group a) and/or wherein said variant is modified by the addition, deletion or substitution of one or more amino acid residues in any one or more of the above sequences and/or wherein said variant fragment retains or has enhanced or comparable immunogenicity when compared to the immunogenicity of any one or more of the variants in group a) and/or group b).

7. A vector or DNA vaccine comprising a nucleic acid molecule encoding a DnaJ heat shock protein family (Hsp40) member B7 (termed DNAJB7), or at least one immunogenic fragment thereof, according to claim 1.

8. The vector or DNA vaccine according to claim 7 wherein said nucleic acid molecule is part of, or provided in, an expression vector adapted to express said DNAJB7, or at least one of said fragments thereof.

9. The vector or DNA vaccine according to claim 8 wherein said adaptation includes the provision of at least one transcription control sequences which mediate(s) said expression.

10. The vector or DNA vaccine according to claim 9 wherein the at least one transcription control sequence(s) is/are cell/tissue specific and adapted for inducible or constitutive expression of said DNAJB7, or at least one fragment thereof.

11. The vector or DNA vaccine according to claim 7, wherein said nucleic acid molecule encodes the whole of said DNAJB7 and/or a number of fragments thereof.

12. A pharmaceutical composition comprising the immunogenic agent according to claim 1.

13. (canceled)

14. (canceled)

15. A method of vaccinating a subject suffering from or having a predisposition for cancer comprising administering an effective amount of the immunogenic agent according to claim 1.

16. The method of claim 15, wherein said cancer is selected from any one or more of the following cancers: nasopharyngeal cancer, synovial cancer, hepatocellular cancer, renal cancer, cancer of connective tissues, melanoma, lung cancer, bowel cancer, colon cancer, rectal cancer, colorectal cancer, brain cancer, throat cancer, oral cancer, liver cancer, bone cancer, pancreatic cancer, choriocarcinoma, gastrinoma, pheochromocytoma, prolactinoma, T-cell leukemia/lymphoma, tonsil, spleen, neuroma, von Hippel-Lindau disease, Zollinger-Ellison syndrome, adrenal cancer, anal cancer, bile duct cancer, bladder cancer, ureter cancer, glioma, oligodendroglioma, neuroblastoma, meningioma, spinal cord tumor, bone cancer, osteochondroma, chondrosarcoma, Ewing's sarcoma, cancer of unknown primary site, carcinoid, carcinoid of gastrointestinal tract, fibrosarcoma, breast cancer, muscle cancer, Paget's disease, cervical cancer, rectal cancer, esophagus cancer, gall bladder cancer, cholangioma cancer, head cancer, eye cancer, nasopharynx cancer, neck cancer, kidney cancer, Wilms' tumor, liver cancer, Kaposi's sarcoma, prostate cancer, testicular cancer, Hodgkin's disease, non-Hodgkin's lymphoma, skin cancer, mesothelioma, myeloma, multiple myeloma, ovarian cancer, endocrine pancreatic cancer, glucagonoma, parathyroid cancer, penis cancer, pituitary cancer, soft tissue sarcoma, retinoblastoma, small intestine cancer, stomach cancer, thymus cancer, thyroid cancer, trophoblastic cancer, hydatidiform mole, uterine cancer, endometrial cancer, vagina cancer, vulva cancer, acoustic neuroma, mycosis fungoides, insulinoma, carcinoid syndrome, somatostatinoma, gum cancer, heart cancer, lip cancer, meninges cancer, mouth cancer, nerve cancer, palate cancer, parotid gland cancer, peritoneum cancer, pharynx cancer, pleural cancer, salivary gland cancer, tongue cancer and tonsil cancer.

17. The method according to claim 16, wherein said cancer is selected from the group comprising the following cancers: colorectal cancer, thyroid, lymphoma, lung, liver, pancreatic, carcinoid, head & neck, stomach, urothelial, prostate, testis, endometrial, glioma, breast, cervical, ovarian, melanoma, liver, and renal cancers.

18. A pharmaceutical composition comprising the vector or DNA vaccine according to claim 7.

19. A method of vaccinating a subject suffering from or having a predisposition for cancer, comprising administering the vector or DNA vaccine according to claim 7.

20. A method of vaccinating a subject suffering from or having a predisposition for cancer, comprising administering the pharmaceutical composition according to claim 12 to said subject.

21. The method of claim 19, wherein said cancer is selected from any one or more of the following cancers: nasopharyngeal cancer, synovial cancer, hepatocellular cancer, renal cancer, cancer of connective tissues, melanoma, lung cancer, bowel cancer, colon cancer, rectal cancer, colorectal cancer, brain cancer, throat cancer, oral cancer, liver cancer, bone cancer, pancreatic cancer, choriocarcinoma, gastrinoma, pheochromocytoma, prolactinoma, T-cell leukemia/lymphoma, tonsil, spleen, neuroma, von Hippel-Lindau disease, Zollinger-Ellison syndrome, adrenal cancer, anal cancer, bile duct cancer, bladder cancer, ureter cancer, glioma, oligodendroglioma, neuroblastoma, meningioma, spinal cord tumor, bone cancer, osteochondroma, chondrosarcoma, Ewing's sarcoma, cancer of unknown primary site, carcinoid, carcinoid of gastrointestinal tract, fibrosarcoma, breast cancer, muscle cancer, Paget's disease, cervical cancer, rectal cancer, esophagus cancer, gall bladder cancer, cholangioma cancer, head cancer, eye cancer, nasopharynx cancer, neck cancer, kidney cancer, Wilms' tumor, liver cancer, Kaposi's sarcoma, prostate cancer, testicular cancer, Hodgkin's disease, non-Hodgkin's lymphoma, skin cancer, mesothelioma, myeloma, multiple myeloma, ovarian cancer, endocrine pancreatic cancer, glucagonoma, parathyroid cancer, penis cancer, pituitary cancer, soft tissue sarcoma, retinoblastoma, small intestine cancer, stomach cancer, thymus cancer, thyroid cancer, trophoblastic cancer, hydatidiform mole, uterine cancer, endometrial cancer, vagina cancer, vulva cancer, acoustic neuroma, mycosis fungoides, insulinoma, carcinoid syndrome, somatostatinoma, gum cancer, heart cancer, lip cancer, meninges cancer, mouth cancer, nerve cancer, palate cancer, parotid gland cancer, peritoneum cancer, pharynx cancer, pleural cancer, salivary gland cancer, tongue cancer and tonsil cancer.

22. The method according to claim 21, wherein said cancer is selected from the group comprising the following cancers: colorectal cancer, thyroid, lymphoma, lung, liver, pancreatic, carcinoid, head & neck, stomach, urothelial, prostate, testis, endometrial, glioma, breast, cervical, ovarian, melanoma, liver, and renal cancers.

23. The method according to claim 20, wherein said cancer is selected from any one or more of the following cancers: nasopharyngeal cancer, synovial cancer, hepatocellular cancer, renal cancer, cancer of connective tissues, melanoma, lung cancer, bowel cancer, colon cancer, rectal cancer, colorectal cancer, brain cancer, throat cancer, oral cancer, liver cancer, bone cancer, pancreatic cancer, choriocarcinoma, gastrinoma, pheochromocytoma, prolactinoma, T-cell leukemia/lymphoma, tonsil, spleen, neuroma, von Hippel-Lindau disease, Zollinger-Ellison syndrome, adrenal cancer, anal cancer, bile duct cancer, bladder cancer, ureter cancer, glioma, oligodendroglioma, neuroblastoma, meningioma, spinal cord tumor, bone cancer, osteochondroma, chondrosarcoma, Ewing's sarcoma, cancer of unknown primary site, carcinoid, carcinoid of gastrointestinal tract, fibrosarcoma, breast cancer, muscle cancer, Paget's disease, cervical cancer, rectal cancer, esophagus cancer, gall bladder cancer, cholangioma cancer, head cancer, eye cancer, nasopharynx cancer, neck cancer, kidney cancer, Wilms' tumor, liver cancer, Kaposi's sarcoma, prostate cancer, testicular cancer, Hodgkin's disease, non-Hodgkin's lymphoma, skin cancer, mesothelioma, myeloma, multiple myeloma, ovarian cancer, endocrine pancreatic cancer, glucagonoma, parathyroid cancer, penis cancer, pituitary cancer, soft tissue sarcoma, retinoblastoma, small intestine cancer, stomach cancer, thymus cancer, thyroid cancer, trophoblastic cancer, hydatidiform mole, uterine cancer, endometrial cancer, vagina cancer, vulva cancer, acoustic neuroma, mycosis fungoides, insulinoma, carcinoid syndrome, somatostatinoma, gum cancer, heart cancer, lip cancer, meninges cancer, mouth cancer, nerve cancer, palate cancer, parotid gland cancer, peritoneum cancer, pharynx cancer, pleural cancer, salivary gland cancer, tongue cancer and tonsil cancer.

24. The method according to claim 23, wherein said cancer is selected from the group comprising the following cancers: colorectal cancer, thyroid, lymphoma, lung, liver, pancreatic, carcinoid, head & neck, stomach, urothelial, prostate, testis, endometrial, glioma, breast, cervical, ovarian, melanoma, liver, and renal cancers.

25. A method of vaccinating a subject suffering from or having a predisposition for cancer, comprising administering the pharmaceutical composition according to claim 18 to said subject.

26. The method according to claim 25, wherein said cancer is selected from any one or more of the following cancers: nasopharyngeal cancer, synovial cancer, hepatocellular cancer, renal cancer, cancer of connective tissues, melanoma, lung cancer, bowel cancer, colon cancer, rectal cancer, colorectal cancer, brain cancer, throat cancer, oral cancer, liver cancer, bone cancer, pancreatic cancer, choriocarcinoma, gastrinoma, pheochromocytoma, prolactinoma, T-cell leukemia/lymphoma, tonsil, spleen, neuroma, von Hippel-Lindau disease, Zollinger-Ellison syndrome, adrenal cancer, anal cancer, bile duct cancer, bladder cancer, ureter cancer, glioma, oligodendroglioma, neuroblastoma, meningioma, spinal cord tumor, bone cancer, osteochondroma, chondrosarcoma, Ewing's sarcoma, cancer of unknown primary site, carcinoid, carcinoid of gastrointestinal tract, fibrosarcoma, breast cancer, muscle cancer, Paget's disease, cervical cancer, rectal cancer, esophagus cancer, gall bladder cancer, cholangioma cancer, head cancer, eye cancer, nasopharynx cancer, neck cancer, kidney cancer, Wilms' tumor, liver cancer, Kaposi's sarcoma, prostate cancer, testicular cancer, Hodgkin's disease, non-Hodgkin's lymphoma, skin cancer, mesothelioma, myeloma, multiple myeloma, ovarian cancer, endocrine pancreatic cancer, glucagonoma, parathyroid cancer, penis cancer, pituitary cancer, soft tissue sarcoma, retinoblastoma, small intestine cancer, stomach cancer, thymus cancer, thyroid cancer, trophoblastic cancer, hydatidiform mole, uterine cancer, endometrial cancer, vagina cancer, vulva cancer, acoustic neuroma, mycosis fungoides, insulinoma, carcinoid syndrome, somatostatinoma, gum cancer, heart cancer, lip cancer, meninges cancer, mouth cancer, nerve cancer, palate cancer, parotid gland cancer, peritoneum cancer, pharynx cancer, pleural cancer, salivary gland cancer, tongue cancer and tonsil cancer.

27. The method according to claim 26, wherein said cancer is selected from the group comprising the following cancers: colorectal cancer, thyroid, lymphoma, lung, liver, pancreatic, carcinoid, head & neck, stomach, urothelial, prostate, testis, endometrial, glioma, breast, cervical, ovarian, melanoma, liver, and renal cancers.


SEQUENCE LISTING (32 sequences)

SEQ ID NO: 1 - protein, 20 aa, Homo sapiens
MVDYYEVLGLQRYASPEDIK

SEQ ID NO: 2 - protein, 20 aa, Homo sapiens
QRYASPEDIKKAYHKVALKW

SEQ ID NO: 3 - protein, 20 aa, Homo sapiens
KAYHKVALKWHPDKNPENKE

SEQ ID NO: 4 - protein, 20 aa, Homo sapiens
HPDKNPENKEEAERKFKEVA

SEQ ID NO: 5 - protein, 20 aa, Homo sapiens
EAERKFKEVAEAYEVLSNDE

SEQ ID NO: 6 - protein, 20 aa, Homo sapiens
EAYEVLSNDEKRDIYDKYGT

SEQ ID NO: 7 - protein, 20 aa, Homo sapiens
KRDIYDKYGTEGLNGGGSHF

SEQ ID NO: 8 - protein, 20 aa, Homo sapiens
EGLNGGGSHFDDECEYGFTF

SEQ ID NO: 9 - protein, 20 aa, Homo sapiens
DDECEYGFTFHKPDDVFKEI

SEQ ID NO: 10 - protein, 20 aa, Homo sapiens
HKPDDVFKEIFHERDPFSFH

SEQ ID NO: 11 - protein, 20 aa, Homo sapiens
FHERDPFSFHFFEDSLEDLL

SEQ ID NO: 12 - protein, 20 aa, Homo sapiens
FFEDSLEDLLNRPGSSYGNR

SEQ ID NO: 13 - protein, 20 aa, Homo sapiens
NRPGSSYGNRNRDAGYFFST

SEQ ID NO: 14 - protein, 20 aa, Homo sapiens
NRDAGYFFSTASEYPIFEKF

SEQ ID NO: 15 - protein, 20 aa, Homo sapiens
ASEYPIFEKFSSYDTGYTSQ

SEQ ID NO: 16 - protein, 20 aa, Homo sapiens
SSYDTGYTSQGSLGHEGLTS

SEQ ID NO: 17 - protein, 20 aa, Homo sapiens
GSLGHEGLTSFSSLAFDNSG

SEQ ID NO: 18 - protein, 20 aa, Homo sapiens
FSSLAFDNSGMDNYISVTTS

SEQ ID NO: 19 - protein, 20 aa, Homo sapiens
MDNYISVTTSDKIVNGRNIN

SEQ ID NO: 20 - protein, 20 aa, Homo sapiens
DKIVNGRNINTKKIIESDQE

SEQ ID NO: 21 - protein, 20 aa, Homo sapiens
TKKIIESDQEREAEDNGELT

SEQ ID NO: 22 - protein, 20 aa, Homo sapiens
REAEDNGELTFFLVNSVANE

SEQ ID NO: 23 - protein, 20 aa, Homo sapiens
FFLVNSVANEEGFAKECSWR

SEQ ID NO: 24 - protein, 20 aa, Homo sapiens
EGFAKECSWRTQSFNNYSPN

SEQ ID NO: 25 - protein, 20 aa, Homo sapiens
TQSFNNYSPNSHSSKHVSQY

SEQ ID NO: 26 - protein, 20 aa, Homo sapiens
SHSSKHVSQYTFVDNDEGGI

SEQ ID NO: 27 - protein, 20 aa, Homo sapiens
TFVDNDEGGISWVTSNRDPP

SEQ ID NO: 28 - protein, 20 aa, Homo sapiens
SWVTSNRDPPIFSAGVKEGG

SEQ ID NO: 29 - protein, 20 aa, Homo sapiens
IFSAGVKEGGKRKKKKRKEV

SEQ ID NO: 30 - protein, 19 aa, Homo sapiens
KRKKKKRKEVQKKSTKRNC

SEQ ID NO: 31 - protein, 309 aa, Homo sapiens
MVDYYEVLGLQRYASPEDIKKAYHKVALKWHPDKNPENKEEAERKFKEVAEAYEVLSNDEKRDIYDKYGTEGLNGGGSHFDDECEYGFTFHKPDDVFKEIFHERDPFSFHFFEDSLEDLLNRPGSSYGNRNRDAGYFFSTASEYPIFEKFSSYDTGYTSQGSLGHEGLTSFSSLAFDNSGMDNYISVTTSDKIVNGRNINTKKIIESDQEREAEDNGELTFFLVNSVANEEGFAKECSWRTQSFNNYSPNSHSSKHVSQYTFVDNDEGGISWVTSNRDPPIFSAGVKEGGKRKKKKRKEVQKKSTKRNC

SEQ ID NO: 32 - DNA, 2578 bp, Homo sapiens
taacattgtctttaacataaaagtgtacctatttgcagtcactacctctatcaccaccaccagcagagcctgagctgaggaaaccacggttctcaatacccagcacacccacttccaactatctgttaaaacatggtggattactatgaagttctaggactgcaaagatatgcttcacctgaggacattaaaaaagcttatcataaagtggcacttaaatggcaccctgataaaaatccagaaaataaagaagaagcagagagaaaattcaaagaagtagctgaggcatacgaggtattatcaaatgatgagaaacgggacatttatgataaatatggcacagaaggattaaacggaggtggaagtcattttgatgatgaatgtgagtacggcttcacattccataagccagatgatgtttttaaagaaatttttcatgaaagggatccattttcttttcacttctttgaagactcgcttgaggacctgttaaatcgtccaggaagctcctatggaaacagaaacagagatgcaggatactttttctccactgccagtgaatatccaatttttgagaaattttcttcatatgatacaggatatacatcacagggttcattggggcatgaaggccttacttctttctcttccctggcttttgataatagtgggatggacaactacatatctgttacaacttcagacaaaatcgttaatggcagaaatattaatacaaagaaaattattgaaagtgatcaagaaagagaagctgaagataatggagagttgacattttttcttgtaaatagtgtggccaatgaagagggctttgcaaaagaatgcagctggagaacacagtcattcaacaactattcaccaaattctcacagctccaaacatgtatctcaatatactttcgtggacaatgatgagggaggtatatcttgggttaccagcaacagagatccccctattttctcagcaggagtcaaagagggtggtaagaggaaaaaaaagaagcgtaaagaggtgcaaaagaagtctaccaaaaggaattgttaaattgactcttcaaatatataacatttgaacacaattgtgtgtgttttggttaatcacaaattttgtagataacacttaatactatactaagagcttttcaacacttttagcaggattgtggacatttggttagtagtttttttgaatgggtatgtcagaaaaggatgagtttgtggtgacagttggtgctaataagaatttgcctgggcagtatagtgagatcttttctctacaaaaaatttaaaaattagccaagtgtggtggtgtgcacctgtagtcccagctactcgggaagctggtagaaagactgcttgagcctggaagacataggttgcagtgtaatgggatcacgccactgcactccagcttgggtgacaaagtaagaccctgtcttaaaaaaaaaaaaaaggaatttggaaaactgcaatgagttttttacttttgaatttttctttagtagagtttatagcctatttttcttttttctaattaaaaccactatacaaacttacgaaacatgcatagcattagaagacaagaatgtatcaagacacacaggttcagactacacaattattcatgaaatggctgttttccaatggaggaacagcatttgatgcattataacttaaaggtactacaaattaaaaattaaagtataaagtattttaagttagaaatagacttagctgggtgcagtggctcacacctgtaatcctagcatcttgggaggcccaggcaggagggttgaagtcagaatttcacgacagtccgggtaacataatgagacctgtcctctatgaaaaaacttttttaaaaatttagctgggtgtggtggtacgtgcctgtaggcctagctacttaggaggctgaggtgggaggattgcttgagcccaggagtttgaggttgtagggagctatgatcttgccattgcactccagctgagagtgtgaccctgtctctaaagaaaaactatagaaaatttaaaaaattaaaaaacgggtaacatggtaaattttatgtagtatattttaccacattttttttaaaggccagtcttatcctgaattcctcagagcagtctgtatggtaggccatcagtgaatcactccagacaggaaattttctatttttgtttagagtttgtacacaaaatctacaaatacattactctaaaatagcacctatagccattttaatttagtcatgttgggagaaaaatagatgggttacaaatacatcagatatcgagcaaatggtttgagttacatctagcagcactaaaacttattcaactttggccaaatacaaatgcgaaattagaaaaaaattataatatctgctatttttaaaagacagctatagtcaagagcttgtgttttgtatcggtatttgatttttagtgggcagatgggagaaggaaaggttatttattctatagaagagactgcggacaattagtattcaaagtttattttcagaaaataaaataataaaagcccagggtattttaaa